(19) United States
(12) Patent Application Publication
(10) Pub. No.: US 2021/0228806 A1
(43) Pub. Date: Jul. 29, 2021

(54) LOW-PROFILE MULTI-AGENT INJECTION SYSTEM AND METHODS

(71) Applicant: PRESAGE BIOSCIENCES, INC., Seattle, WA (US)

(72) Inventors: John STREETER, Seattle, WA (US); Jason FRAZIER, Seattle, WA (US); Robert W. HENSON, Morrisville, NC (US); John M. DONOHOE, Morrisville, NC (US); Edward P. BROWKA, Morrisville, NC (US); Eli B. NICHOLS, Morrisville, NC (US); Theodore J. MOSLER, Morrisville, NC (US)

(21) Appl. No.: 17/059,994

(22) PCT Filed: May 31, 2019
(86) PCT No.: PCT/US19/34912

§ 371 Date: Nov. 30, 2020

Related U.S. Application Data
(60) Provisional application No. 62/679,589, filed on Jun. 1, 2018.

(51) Int. Cl. A61M 5/178; A61M 5/19; A61M 5/20; A61M 5/315

(52) U.S. Cl. CPC A61M 5/1782; A61M 5/19; A61M 5/2033; A61M 2005/005; A61M 5/31581; A61M 5/31596; A61M 5/2066

(57) ABSTRACT

Low-profile fluid injection systems and methods for injecting fluid into a tumor within a body comprising an elongate member, a plurality of fluid delivery members, and a plurality of fluid reservoirs. Each of the plurality of fluid reservoirs may be fluidly coupled to a single of fluid delivery lumen of a single fluid delivery member such that each of the fluid delivery members is fluidly independent of every other fluid delivery member. The plurality of fluid delivery members may be configured to extend out of a distal end of the elongate member into the tumor when the system is positioned in the body of a patient. A fluid delivery mechanism may be operably coupled to the plurality of fluid reservoirs in order to deliver fluids from the plurality of fluid reservoirs to the plurality of fluid delivery members and inject the fluids into the tumor.

### CROSS-REFERENCE

This application claims the benefit of U.S. Provisional Application No. 62/679,589, filed Jun. 1, 2018, which is incorporated herein by reference in its entirety for all purposes.

### BACKGROUND

A fundamental problem in cancer drug development is that antitumor efficacy in preclinical cancer models may not translate to efficacy in patients or patient outcome. In many instances, drugs may be tested in preclinical in vitro or in vivo systems that fail to accurately represent the clinical disease. In vitro cell-culture based systems for example often provide static, homogenous testing conditions which cannot take into account the effects of changing microenvironmental conditions or cellular heterogeneity of tumors, for example. In vivo animal model based systems may provide somewhat better translation to the clinic, but they are often hampered in their predictive usefulness by differences in tumor microenvironments (particularly genetic, molecular, immunologic, and cellular difference), varied growth conditions, and a variety of other factors compared to clinical human tumors.

### SUMMARY

It would therefore be desirable to provide improved methods, systems, and devices for drug candidate testing for efficacy in clinical tumors. Proposed embodiments of such a system may provide for in situ injection of one or more drug candidates into discrete, mapped locations of a clinical tumor for simultaneous assessment of the drug candidates in the growing tumor of a living subject. The effect of the drugs may be observed as spatially defined tumor responses following resection or biopsy of the injected tumor tissue. In this way, the efficacy of multiple drug candidates may be assessed directly in the clinical setting, which may lead to improved prediction of therapeutic response to systemic drug delivery.

Moreover, it would also be desirable if improved methods, systems, and devices for drug candidate testing were able to reach subdermal tumors in a minimally-invasive manner. Proposed embodiments of such a system may for example be compatible with existing non- or minimally-invasive surgical access devices or introducers such as biopsy instrumentation, laparoscopic equipment, endovascular catheters, or the like. Alternatively or in combination, embodiments of such a system may comprise its own introducer to provide access to the tumor site of interest. The systems described herein may be configured to access superficial tumors and/or those tumors which are less accessible and/or located deeper inside the body.

It would also be desirable if improved methods, systems, and devices for drug candidate testing allowed for simplified loading of drug candidates. Proposed embodiments of such a system may for example include one or more cartridges containing one or more drug candidates therein. Each cartridge may be pre-loaded with a drug candidate and configured for insertion into a delivery system which delivers the drug candidate from the cartridge to the tumor site of interest.

At least some of these objectives are met by the exemplary embodiments described below. Not necessarily all such aspects or advantages are achieved by any particular embodiment. Thus, various embodiments may be carried out in a manner that achieves or optimizes one advantage or group of advantages taught herein without necessarily achieving other aspects or advantages as may also be taught or suggested herein.

The present disclosure generally relates to medical devices, systems, and methods, and more particularly relates to methods and apparatus used to inject one or more fluids, for example one or more drug candidates or combinations, into a tissue.

An aspect of the present disclosure provides a fluid injection system. In some embodiments, the fluid injection system comprises an elongate member having a proximal end and a distal end. In some embodiments, the elongate member comprises an inner wall defining a lumen therein. In some embodiments, the fluid injection system comprises a plurality of fluid delivery members. In some cases, the plurality of fluid delivery members is disposed within the lumen of the elongate member. In some cases, the plurality of fluid delivery members has a retracted configuration and an extended configuration. In some embodiments, the plurality of fluid delivery members is configured to extend out of the distal end of the elongate member in the extended configuration. In some embodiments, each of the plurality of fluid delivery members comprises a distal end, a proximal end, an outlet port at the distal end, and an inner wall defining a fluid delivery lumen therein. In some embodiments, the fluid delivery lumen is fluidly coupled to the outlet port. In some embodiments, each of the fluid delivery lumens is fluidly independent of every other fluid delivery lumen of every other fluid delivery lumen of the plurality of fluid delivery members. In some embodiments, the fluid injection system comprises a plurality of fluid delivery channels. In some embodiments, each of the plurality of fluid delivery channels is fluidly coupled to one or more fluid delivery lumens of the plurality of fluid delivery members. In some embodiments, a fluid delivery mechanism is operably coupled to the plurality of fluid delivery channels, wherein actuation of the fluid delivery mechanism causes fluid to pass from the plurality of fluid delivery channels to the plurality of fluid delivery members and out of the outlet ports.

In some embodiments, actuation of the fluid delivery mechanism is operably coupled to the plurality of fluid delivery members such that delivery of fluid is concomitant with retraction of the fluid delivery members from the extended to the retracted configuration. In some embodiments, the fluid delivery mechanism comprises a fluid delivery rod. In some embodiments, the plurality of fluid delivery members is configured to retract from the extended configuration to the retracted configuration simultaneously with fluid delivery from the fluid delivery members. In some embodiments, the plurality of fluid delivery members is configured to be fully enclosed within the lumen of the elongate member in the retracted configuration.

In some embodiments, the elongate member comprises a sheath, a hypotube shaft, or a needle. In some embodiments, the elongate member comprises a metal. In some embodiments, the elongate member comprises a flexible material. In some embodiments, the elongate member comprises a rigid material. In some embodiments, the elongate member has a length in a range from about 4 cm to about 250 cm. In some embodiments, the elongate member has a length in a range from about 4 cm to about 20 cm. In some embodiments, the elongate member has a length in a range of about 4 cm to about 20 cm. In some embodiments, the elongate member has a length in a range of about 100 cm to about 250 cm. In some embodiments, the elongate member has an outer diameter in a range of about 0.9 mm to about 3.5 mm. In some embodiments, the elongate member has an outer diameter in a range of about 2 mm to about 4 mm. In some embodiments, the elongate member has an outer diameter in a range of about 3 French to about 10 French. In some embodiments, the elongate member has an outer diameter sized to fit within a working channel of a conventional biopsy access needle, a conventional endoscope, or a conventional vascular access sheath. In some embodiments, the elongate member comprises a needle with a gauge number in a range of about 10 to about 20.

In some embodiments, the plurality of fluid delivery members comprises at least two fluid delivery members. In some embodiments, the plurality of fluid delivery members comprises from 2 to 20 fluid delivery members. In some embodiments, the plurality of fluid delivery members comprises a plurality of needles or tubes. In some embodiments, the plurality of fluid delivery members comprises a plurality of pencil-point needles, blunt-tipped needles, or bevel-tipped needles In some embodiments, the plurality of fluid delivery members comprises metal or plastic. In some embodiments, the plurality of fluid delivery members comprises a shape-memory alloy. In some embodiments, the plurality of fluid delivery members comprises a flexible material. In some embodiments, the plurality of fluid delivery members comprises a rigid material. In some embodiments, each of the plurality of fluid delivery members has an outer diameter in a range of about 0.05 mm to about 0.50 mm. In some embodiments, each of the plurality of fluid delivery members has an outer diameter of about 0.25 mm. In some embodiments, each of the plurality of fluid delivery members is a needle with a gauge number of about 28 to about 33. In some embodiments, each of the plurality of fluid delivery members is a needle with a gauge number of about 31. In some embodiments, each of the fluid delivery lumens of the plurality of fluid delivery members has a volume in a range of about 0.1 μl to about 10 μl. In some embodiments, each of the plurality of fluid delivery members has a length extending from the distal end of the elongate member to the proximal end of the elongate member. In some embodiments, each of the plurality of fluid delivery members has a length in a range of about 4 cm to about 250 cm. In some embodiments, each of the plurality of fluid delivery members has a length extending out of the distal end of the elongate member in the extended configuration in a range of about 5 mm to about 40 mm. In some embodiments, each of the plurality of fluid delivery members comprises at least one additional outlet port fluidly coupled to the fluid delivery lumen. In some embodiments, in the extended configuration, each of the plurality of fluid delivery members angle away from a longitudinal axis of the elongate member. In some embodiments, each of the plurality of fluid delivery members angle away from the longitudinal axis of the elongate member at an angle in a range of about 10° to about 90°.

In some embodiments, the distal end of the elongate member comprises angling elements positioned to guide the plurality of fluid delivery members to angle away from a longitudinal axis of the elongate member in the extended configuration. In some embodiments, in the extended configuration, each of the plurality of fluid delivery members angle away from a longitudinal axis of the elongate members such that a distance between distal ends of each of the plurality of fluid delivery members is in a range of about 1 mm to about 10 mm.

In some embodiments, the system further comprises a handle adjacent to the proximal end of the elongate member.

In some embodiments, the system further comprises an actuator adjacent to the proximal end of the elongate member and operably coupled to the plurality of fluid delivery members, wherein actuation of the actuator moves the plurality of fluid delivery members from the retracted configuration to the expanded configuration or from the expanded configuration to the retracted configuration. In some embodiments, the actuator is configured to retract the plurality of fluid delivery members from the expanded configuration to the retracted configuration at a speed in a range of about 0.1 mm/s to about 10 mm/s. In some embodiments, the actuator comprises a mechanical actuator or an electromechanical actuator. In some embodiments, the actuator is manually operated. In some embodiments, the actuator is automatically operated.

In some embodiments, the fluid delivery mechanism is actuated by the actuator. In some embodiments, the fluid delivery mechanism comprises a mechanical actuator or an electromechanical actuator. In some embodiments, the fluid delivery mechanism comprises one or more of a plunger or a pump. In some embodiments, the fluid delivery mechanism is manually operated. In some embodiments, the fluid delivery mechanism is automatically operated. In some embodiments, the fluid delivery mechanism is configured to cause fluid to be delivered out of the outlet ports at a flow rate in a range of about 0.1 μl/s to about 10 μl/s.

In some embodiments, the system is configured for fluid delivery from about 1 cm to about 300 cm below the skin surface. In some embodiments, the system is configured for fluid delivery from about 1 cm to about 30 cm below the skin surface. In some embodiments, the system is configured for fluid delivery from about 4 cm to about 20 cm below the skin surface. In some embodiments, the system is configured for fluid delivery from about 100 cm to about 250 cm below the skin surface.

In some embodiments, the plurality of fluid delivery channels comprises the fluid delivery lumens of the plurality of fluid delivery members. In some embodiments, the fluid delivery lumens of the plurality of fluid delivery members are the plurality of fluid delivery channels. In some embodiments, the fluid delivery mechanism comprises a plurality of fluid delivery mechanisms, each of the plurality of fluid delivery mechanisms being operably coupled to a single fluid delivery channel of the plurality of fluid delivery channels.

In some embodiments, the system further comprises an imaging system for peri-operative imaging of the fluid injection system in use.

In some embodiments, the system further comprises one or more cartridges fluidly-coupled to one or more of the fluid delivery lumens or one or more of the plurality of fluid delivery channels. In some embodiments, each of the plurality of fluid delivery channels has a volume in a range of about 10 μl to about 500 μl.

In some embodiments, the system further comprises a population of fluorescent tracking microspheres (FTM). In some embodiments, the fluorescent tracking microspheres have a diameter from 5 micrometers to 10 micrometers. In some embodiments, the fluorescent tracking microspheres comprise polystyrene. In some embodiments, the system further comprises a plurality of populations of fluorescent tracking microspheres (FTM).

In some embodiments, the system further comprises a volume selector. In some embodiments, the system further comprises a plurality of cartridges.

An aspect of the present disclosure provides method of injecting fluid into a tumor within a body of a patient, the method comprising: providing a fluid injection system, wherein the fluid injection system comprises an elongate member having a proximal end and a distal end, a plurality of fluid delivery members disposed within a lumen of the elongate member, and a plurality of fluid delivery channels, wherein each of the plurality of fluid delivery channels is fluidly coupled to a single fluid delivery lumen of each of the plurality of fluid delivery members; inserting the distal end of the elongate member into the body with the plurality of fluid delivery members retracted; positioning the distal end of the elongate member in close proximity to the tumor with the plurality of fluid delivery members retracted; extending the plurality of fluid delivery members from the distal end of the elongate member into the tumor; and injecting a plurality of fluids into the tumor from the plurality of fluid delivery members, wherein each of the plurality of fluid delivery members is fluidly independent from every other of the plurality of fluid delivery members.

In some embodiments, the method further comprises retracting the plurality of fluid delivery members from the tumor into the distal end of the elongate member. In some embodiments, retracting the plurality of fluid delivery members occurs concomitantly with injecting the plurality of fluids. In some embodiments, retracting the plurality of fluid delivery members comprises retracting the plurality of fluid delivery members such that the plurality of fluid delivery members is fully enclosed within the lumen of the elongate member. In some embodiments, retracting the plurality of fluid delivery members comprises retracting the plurality of fluid delivery members at a speed in a range of about 0.1 mm/s to about 10 mm/s.

In some embodiments, the method further comprises removing the distal end of the elongate member from the body with the plurality of fluid delivery members retracted. In some embodiments, the method further comprises resecting at least a portion of the tumor for analysis. In some embodiments, the method further comprises loading the plurality of fluids into the plurality of fluid delivery channels prior to inserting the distal end of the elongate member into the body. In some embodiments, the method further comprises imaging the fluid injection system peri-operatively.

In some embodiments, the elongate member comprises a sheath, a hypotube shaft, or a needle. In some embodiments, the elongate member comprises a metal. In some embodiments, the elongate member comprises a flexible material. In some embodiments, the elongate member comprises a rigid material. In some embodiments, the elongate member has an outer diameter in a range of about 0.9 mm to about 3.5 mm. In some embodiments, the elongate member comprises a needle with a gauge number in a range of about 10 to about 20.

In some embodiments, inserting the distal end of the elongate member into the body comprises inserting the distal end of the elongate member into a working channel of a conventional biopsy access needle, a conventional endoscope, or a conventional vascular access sheath pre-positioned in the body.

In some embodiments, the plurality of fluid delivery members comprises at least two fluid delivery members. In some embodiments, the plurality of fluid delivery members comprises from 2 to 20 fluid delivery members. In some embodiments, the plurality of fluid delivery members comprises a plurality of needles or tubes. In some embodiments, the plurality of fluid delivery members comprises metal or plastic. In some embodiments, the plurality of fluid delivery members comprises a shape-memory alloy. In some embodiments, the plurality of fluid delivery members comprises a flexible material. In some embodiments, the plurality of fluid delivery members comprises a rigid material. In some embodiments, each of the plurality of fluid delivery members has an outer diameter of from about 0.05 mm to about 0.50 mm. In some embodiments, each of the plurality of fluid delivery members is a needle with a gauge number of about 28 to about 33.

In some embodiments, injecting the plurality of fluids comprises injecting the plurality of fluids at a flow rate in a range of about 0.1 μl/s to about 10 μl/s. In some embodiments, injecting the plurality of fluids comprises injecting, from each of the plurality of fluid delivery members, a volume in a range of about 10 μl to about 500 μl of each of the plurality of fluids.

In some embodiments, each of the plurality of fluid delivery members has a length extending from the distal end of the elongate member to the proximal end of the elongate member. In some embodiments, each of the plurality of fluid delivery members has a length in a range of about 4 cm to about 250 cm.

In some embodiments, extending the plurality of fluid delivery members comprises extending a length in a range of about 5 mm to about 40 mm of each of the plurality of fluid delivery members out of the distal end of the elongate member into the tumor. In some embodiments, extending the plurality of fluid delivery members comprises extending the plurality of fluid delivery members from the distal end of the elongate member such that the plurality of fluid delivery members angle away from a longitudinal axis of the elongate member.

In some embodiments, the distal end of the elongate member comprises angling elements positioned to guide the plurality of fluid delivery members to angle away from the longitudinal axis of the elongate member in the extended configuration.

In some embodiments, injecting the plurality of fluids comprises creating a plurality of distinct fluid columns in the tumor.

In some embodiments, a fluid injection system used in a method further comprises a handle having a fluid delivery mechanism thereon, the fluid delivery mechanism being operably coupled to the plurality of fluid delivery channels, and wherein injecting the plurality of fluids comprises actuating the fluid delivery mechanism. In some embodiments, the fluid delivery mechanism comprises manually actuating the fluid delivery mechanism. In some embodiments, actuating the fluid delivery mechanism comprises automatically actuating the fluid delivery mechanism. In some embodiments, the fluid delivery mechanism comprises a mechanical actuator or an electromechanical actuator. In some embodiments, the fluid delivery mechanism comprises one or more of a plunger or a pump. In some embodiments, the fluid injection system further comprises an actuator adjacent to the proximal end of the elongate member and operably coupled to the plurality of fluid delivery members. In some embodiments, extending the plurality of fluid delivery members comprises actuating the actuator.

In some embodiments, actuating the actuator comprises manually actuating the actuator. In some embodiments, injecting the plurality of fluids comprises actuating the actuator. In some embodiments, actuating the actuator comprises automatically actuating the actuator. In some embodiments, the actuator comprises a mechanical actuator or an electromechanical actuator. In some embodiments, the actuator comprises one or more of a thumbwheel or an electric actuator. In some embodiments, injecting the plurality of fluids comprises injecting the plurality of fluids from about 0.2 cm to about 20 cm below the skin surface. In some embodiments, injecting the plurality of fluids comprises injecting the plurality of fluids from about 1 cm to about 30 cm below the skin surface. In some embodiments, injecting the plurality of fluids comprises injecting the plurality of fluids from about 4 cm to about 20 cm below the skin surface. In some embodiments, injecting the plurality of fluids comprises injecting the plurality of fluids from about 100 cm to about 250 cm below the skin surface. In some embodiments, the plurality of fluids comprises one or more therapeutic agents. In some embodiments, injecting the plurality of fluids comprises injecting a different fluid from each of the plurality of fluid delivery members into the tumor. In some embodiments, injecting the plurality of fluids comprises injecting a same fluid from each of the plurality of fluid delivery members into the tumor. In some embodiments, the tumor is located in the skin, breast, brain, prostate, colon, rectum, kidney, pancreas, lung, liver, heart, stomach, intestines, ovaries, testes, cervix, lymph nodes, thyroid, esophagus, head or neck, eye, bone, or bladder of the patient. In some embodiments, the plurality of fluids comprises a population of fluorescent tracking microspheres (FTM). In some embodiments, the plurality of fluids comprises a plurality of populations of fluorescent tracking microspheres.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a low-profile fluid injection system, in accordance with embodiments.

FIG. 2 shows a schematic of a low-profile fluid injection system with delivery members extended, in accordance with embodiments.

FIG. 3 shows a schematic of a low-profile fluid injection system, in accordance with embodiments.

FIG. 4A shows a schematic of a portion of a low-profile fluid injection system and cartridges, in accordance with embodiments.

FIG. 4B shows a schematic illustrating the loading of a low-profile fluid injection system with a cartridge, in accordance with embodiments.

FIG. 5A shows a schematic of a low-profile fluid injection system, in accordance with embodiments.

FIG. 5B shows a schematic of a low-profile fluid injection system with delivery member extended, in accordance with embodiments.

FIG. 5C shows a schematic of a portion of the low-profile fluid injection system shown in FIG. 5B, in accordance with embodiments.

FIG. 6A shows a schematic of a low-profile fluid injection system, in accordance with embodiments.

FIG. 6B shows a portion of the low-profile fluid injection system shown in FIG. 6A, in accordance with embodiments.

FIG. 7 shows an image of interior mechanisms of a low-profile fluid injection system, in accordance with embodiments.

FIG. 8 shows a schematic of a low-profile fluid injection system, in accordance with embodiments.

FIG. 9 shows a cross-sectional view of an elongate member of a low-profile fluid injection system, in accordance with embodiments.

FIG. 10A shows a schematic of a low-profile fluid injection system with fluid delivery members in an unextended configuration, in accordance with embodiments.

FIG. 10B shows the system of FIG. 10A with fluid delivery members in an extended configuration, in accordance with embodiments.

FIG. 11A shows exemplary elongate members with fluid delivery members in an unextended configuration, in accordance with embodiments.

FIG. 11B shows exemplary elongate members with fluid delivery members in an extended configuration, in accordance with embodiments.

FIG. 12A shows a distal end of an exemplary elongate member with fluid delivery members in an unextended configuration, in accordance with embodiments.

FIG. 12B shows a distal end of an exemplary elongate member with fluid delivery members in the extended configuration, in accordance with embodiments.

FIG. 13A shows a diagram of a target tissue following injection by a low-profile fluid injection system depicted in cross-section perpendicular to a longitudinal axis of the system, in accordance with embodiments.

FIG. 13B shows a perspective view diagram of injection columns in a target tissue following injection by a low-profile fluid injection system, in accordance with embodiments.

FIG. 13C shows a perspective view diagram of injection columns in a target tissue following injection by a low-profile fluid injection system, in accordance with embodiments.

FIG. 14 shows a schematic of a low-profile fluid injection system with fluid delivery members extended inside of a target tissue, in accordance with embodiments.

FIG. 15A shows an exemplary low-profile fluid injection system with fluid delivery members in an unextended configuration inside of a simulated target tissue, in accordance with embodiments.

FIG. 15B shows the exemplary system of FIG. 15A with fluid delivery members extended into the simulated target tissue, in accordance with embodiments.

FIG. 15C shows the system of FIG. 15A during fluid injection into the simulated target tissue and simultaneous retraction of the fluid delivery members, in accordance with embodiments;

FIG. 16A shows a schematic of a low-profile fluid injection system prior to fluid injection with fluid delivery members in an unextended configuration, in accordance with embodiments.

FIG. 16B shows a schematic of a low-profile fluid injection system prior to fluid injection with fluid delivery members in an extended configuration, in accordance with embodiments.

FIG. 16C shows a schematic of a low-profile fluid injection system after fluid injection with fluid delivery members in an unextended configuration, in accordance with embodiments.

FIG. 16D shows a schematic of a low-profile fluid injection system prior to fluid injection with fluid delivery members in an extended configuration, in accordance with embodiments.

FIG. 16E shows the system of FIG. 16D after simultaneous fluid injection and retraction of the fluid delivery members, in accordance with embodiments.

FIG. 17 shows exemplary steps of a method of injecting fluid into a tumor within a body of a subject using a fluid injection system, in accordance with embodiments.

FIG. 18A shows an image of a low-profile fluid injection system comprising a volume selector, in accordance with embodiments.

FIG. 18B shows an image of a volume selector, in accordance with embodiments.

FIG. 19A shows a schematic of a low-profile fluid injection system comprising a tip cap, in accordance with embodiments.

FIG. 19B shows a schematic of a tip cap, in accordance with embodiments.

FIG. 20A shows a cartridge, in accordance with embodiments.

FIG. 20B shows a cartridge, in accordance with embodiments.

FIG. 21A-FIG. 21D show a method for delivering and detecting one or more agents in a target tissue, in accordance with embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure describes low-profile fluid injection devices and systems and methods of their use. Low-profile fluid injection devices and systems disclosed herein can provide advantages over existing devices, systems, and methods, for example, in diagnostic and/or therapeutic applications. In some cases, low-profile fluid injection systems disclosed herein (e.g., system 100) are used for drug delivery to a cancer in situ. One of skill in the art will appreciate that the devices, systems, and methods disclosed herein may be used in multiple anatomical areas and in multiple surgical procedures. It will also be appreciated by one of skill in the art that insertion of fluid injection systems disclosed herein and/or delivery of one or more agents, as disclosed herein, may be performed by those skilled in subcutaneous injections, such as doctors (e.g., physicians) or non-physician medical professionals (e.g., phlebotomists, clinical technicians, nurses, nurse practitioners, or physician's assistants). The devices may for example be used for pre-clinical, ex vivo, or in vitro drug testing. The methods may be performed on human tissues or tissue samples, or on animal tissue or tissue samples.

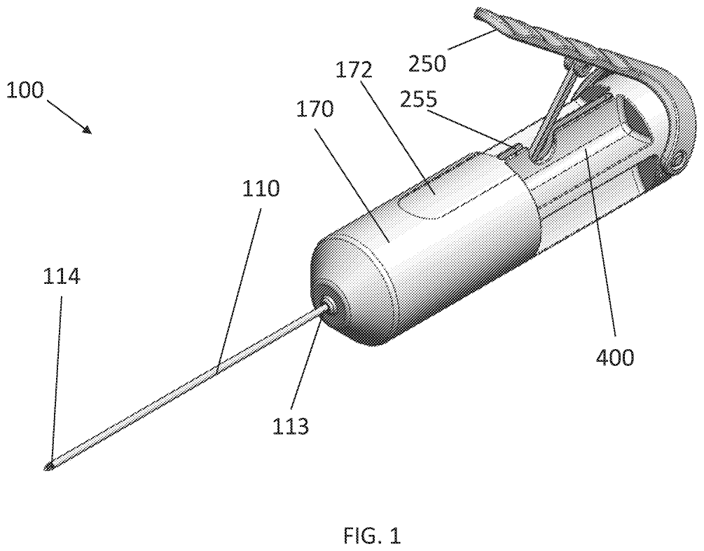

FIG. 1 shows a low-profile fluid injection system 100 comprising actuator 250 and elongate member 110. As will be appreciated by one of skill in the art, the dimensions and structure of low-profile fluid injection systems disclosed herein allow for minimally invasive delivery of one or more fluids (which can comprise, for example, therapeutic and/or diagnostic agents) to a target tissue. As disclosed herein, one or more fluids may be loaded into chamber 400 (e.g., within one or more cartridges 432) and delivered to a target tissue (e.g., a tumor tissue or portion thereof) via one or more fluid delivery members 320 housed within elongate member 110. Elongate member 110 can be connected to the housing of fluid injection system 100 at a proximal end 113 of elongate member 110. In some cases, proximal end 113 of elongate member 110 can comprise distal coupling 190. Distal coupling 190 can be an attachment interface (e.g., a clip or a Luer lock connector). In some cases, a coaxial sheath can be slid over elongate member 110 and coupled to distal coupling 190.

Actuator 250 can be one of various means for driving a syringe body 260 and fluid delivery members 320 within the housing of fluid injection system 100 (which can comprise contoured exterior walls that comprise a hand grip 170 (or handle)). In many cases, actuator 250 comprises a lever arm connected to actuator strut 254, which drives syringe body 260 inside of the housing of fluid injection system 100 via syringe rod 257. In various embodiments, actuator 250 can be manually operated (e.g., by squeezing actuator 250 to the housing of fluid injection system 100). In some cases, actuator 250 may comprise a mechanized actuator, wherein some or all force used in the actuation of syringe body 260 can be provided by an electromechanical mechanism. Actuation of actuator 250 can cause one or more fluid delivery members to extend from a distal end 114 of elongate member 110 (e.g., into a tissue of a subject, such as a target tumor tissue).

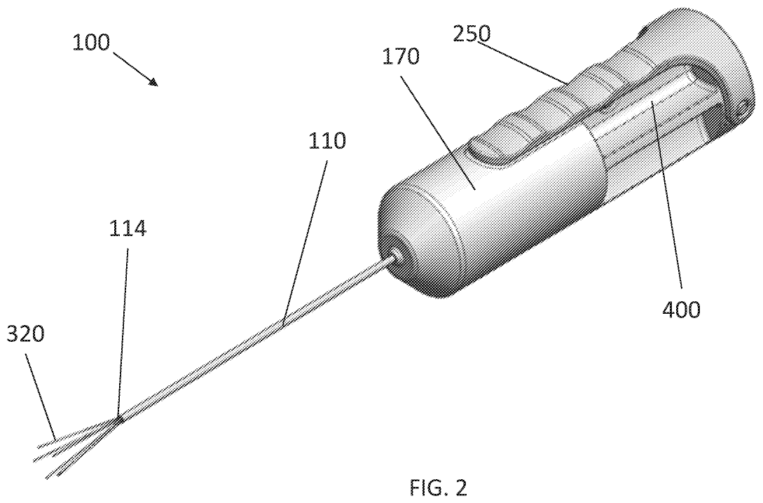

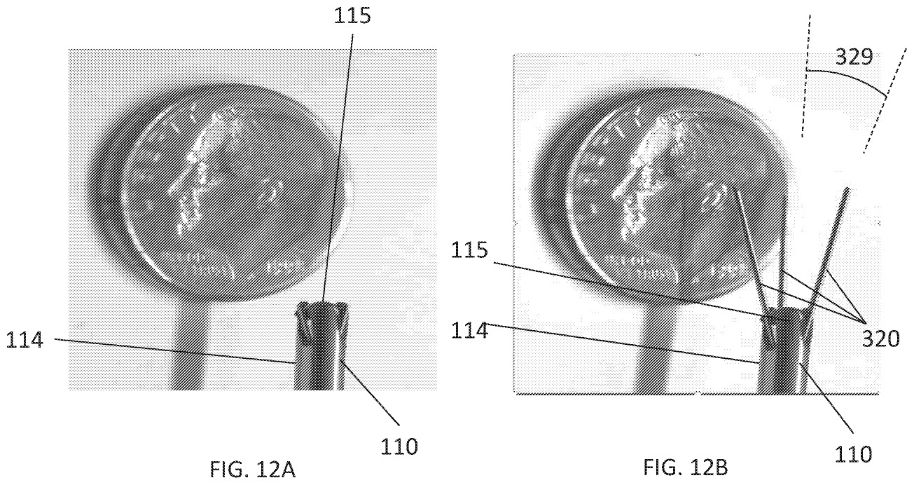

FIG. 2 shows a low-profile fluid injection system 100 with a lever arm of actuator 250 engaged (e.g., depressed). Engaging (e.g., depressing) actuator 250 can cause one or more fluid delivery members to extend out of elongate member 110 via distal end 114 of elongate member 110. Fluid delivery member(s) 320 can be deflected away from a longitudinal axis of fluid injection system 100 (e.g., splayed). A representative example of a plurality of fluid delivery members 320 being splayed as they are extended from distal end 114 of elongate member 110 is shown in FIG. 2. In some cases, distal end 114 of elongate member may comprise one or more angling element 115 (e.g., splaying mechanism) that can cause one or more fluid delivery members 320 to deflect away from a longitudinal axis of fluid injection system 100 when actuator 250 is engaged (e.g., when syringe body 260 is actuated distally within the housing of fluid injection system 100). Angling element 115 can comprise one or more guides, which can comprise angled channel(s) through which fluid delivery members 320 can pass. Representative examples of angling elements 115 are shown in FIG. 12A and FIG. 12B. In some embodiments, one or more fluid delivery member(s) 320 may extend in-line with a longitudinal axis of fluid injection system 100 when actuator 250 is engaged. In some cases wherein a fluid delivery member 320 extends in-line with a longitudinal axis of fluid injection system 100 when actuator 250 is engaged, distal end 114 of elongate member 110 comprises a guide element (e.g., through which fluid delivery member 320 can pass) that is not angled.

In some cases, distal end 114 is shaped to penetrate (e.g., puncture) a tissue. For example, distal end 114 can have a pointed or sharp end, e.g., to penetrate skin or fibrous tissue. In many cases, distal end 114 can have a bullet-shaped or rounded end. Such a bullet-shaped or rounded end of distal end 114 may be sufficient to penetrate skin or fibrous tissue; however, a bullet-shaped or rounded distal end 114 may be advantageous for advancing elongate member through tissues internal to a tissue or subject, as it may avoid damaging (e.g., puncturing) other tissues, such as internal organs. In some cases, a guide element, such as angling element 115 can be shaped to aid in penetration of elongate member 110 into or through a tissue.

In some cases, a lever arm of actuator 250 may be placed in registration with (e.g., in contact with) lever arm recess 172 of the housing of fluid injection system 100, e.g., by engaging actuator 250 completely). Lever arm recess 172 can allow actuator 250 to be depressed to a position more flush with the surface of the housing of fluid injection system 100, which can help a user of fluid injection system 100 to maintain steady control over fluid injection system 100 during use. A representative example of lever arm recess 172 is shown in FIG. 1.

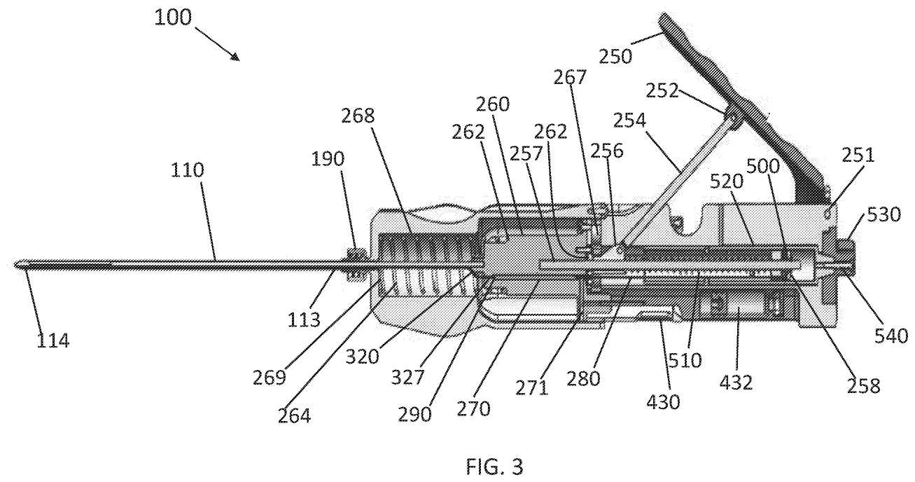

FIG. 3 shows a cross-sectional image of a low-profile fluid injection system 100. Actuator 250 of low-profile fluid injection system 100 can comprise actuator coupling 252. Actuator coupling 252 can be coupled to actuator strut 254 (e.g., rotatably coupled, for example, wherein actuator coupling 252 is a hinge joint). Actuator strut 254 can be coupled to strut coupling 256 (e.g., rotatably coupled, for example, wherein strut coupling 256 is a hinge joint). Strut coupling 256 can be coupled to syringe rod 257 and/or syringe body 260. In some cases, strut coupling is fixedly attached to syringe rod 257 and/or syringe body 260, e.g., wherein rotation or translation between strut coupling 256 and syringe rod 257, syringe body 260, or both syringe rod 257 and syringe body 260 is not permitted.

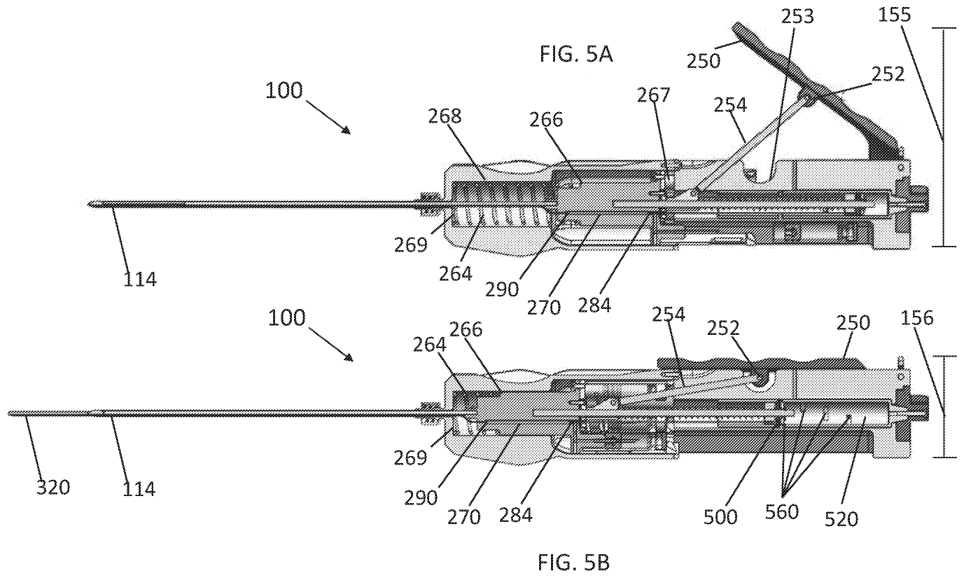

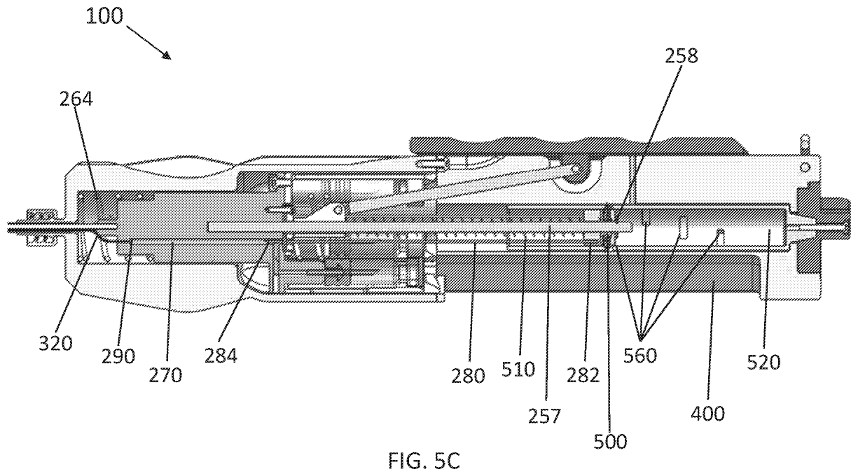

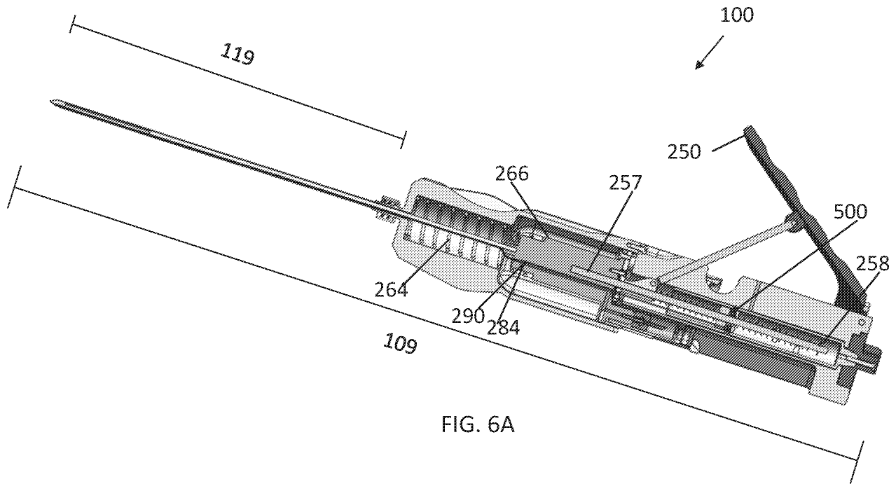

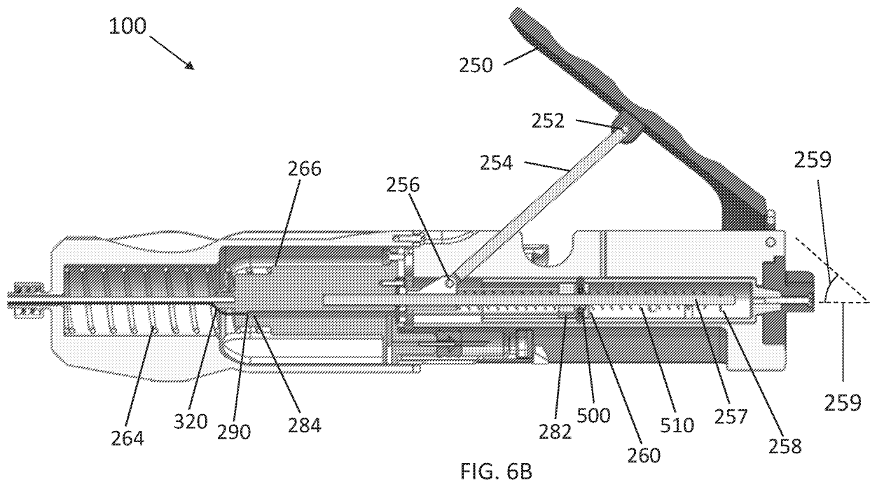

Engaging actuator 250 (e.g., depressing a lever arm of actuator 250) can cause strut 254 to apply force to syringe body 260 (e.g., via syringe rod 257, in some cases), which can cause syringe body 260 to translate slidably through an interior portion of fluid injection system 100 (e.g., through syringe body shaft 268), for example, in a distal direction along a longitudinal axis of fluid injection system 100 (see, e.g., FIG. 5A, FIG. 5B, and FIG. 5C). In some cases, disengaging actuator 250 (e.g., releasing a lever arm of actuator 250) can allow syringe body to translate in a proximal direction along a longitudinal axis of fluid injection system 100 (see, e.g., FIG. 6A and FIG. 6B). In some cases, engaging actuator 250 can cause syringe rod 257 to translate slidably through an interior portion of fluid injection system 100 (e.g., syringe rod shaft 520), e.g., in a distal direction along a longitudinal axis of fluid injection system 100. In some cases, disengaging actuator 250 (e.g., releasing a lever arm of actuator 250) can allow syringe rod 257 to translate fully or partially through an interior portion of fluid injection system 100, e.g., in a proximal direction along a longitudinal axis of fluid injection system 100. A lever arm of actuator 250 can be coupled (e.g., rotatably coupled) to the housing of fluid injection system 100 by actuator hinge 251. In some cases, actuation of actuator 250 causes a lever arm of actuator 250 to rotate around an actuator hinge 251.

In some cases, the housing of fluid injection system 100 can comprise a strut channel 255 to allow actuator strut 254 to move along a longitudinal axis (e.g., during actuation of actuator 250). The housing of fluid injection system 100 can comprise actuator coupling cutout 253. In some cases, actuator coupling cutout 253 is shaped and positioned in the housing of fluid injection system 100 to receive actuator coupling 242 (e.g., the lever arm of actuator 250 is depressed). In some cases, actuator coupling cutout 253 can allow actuator coupling 252 to move inside of a maximum radius of the housing of fluid injection system 100 (e.g., which can allow actuator 250 to be depressed to the point that it is flush with or in contact with an exterior surface of the housing of fluid injection system 100).

One or more fluid delivery members can be coupled to syringe body 260. Translation of syringe body 260 through syringe body shaft 268 can cause one or more fluid delivery members 320 to translate distally through elongate member 110. In some cases, the distance that syringe body 260 and/or one or more fluid delivery members 320 translate in a distal or proximal direction along a longitudinal axis of fluid injection can depend on the degree to which actuator 250 is engaged (e.g., depressed) or unengaged (e.g., released). In some cases, engaging actuator 250 causes one or more fluid delivery members 320 to extend distally from distal end 114 of elongate member 110.

Syringe body spring 264 can be used to resist distal translation of syringe body 260 along a longitudinal axis of fluid injection system 100. In some cases, syringe body spring 264 is disposed between a distal end 269 of syringe body shaft 268 and a shoulder 266 of syringe body 260. Actuation of actuator 250 (e.g., engaging actuator 250, for example, by depressing a lever arm of actuator 250) can cause compression of syringe body spring 264 (e.g., by translating syringe body 260 such that syringe body shoulder 266 is brought closer to distal end 269 of syringe body shaft 268). Disengaging actuator 250 (e.g., releasing a lever arm of actuator 250) can allow syringe body spring to extend and can cause syringe body 260 to translate proximally along a longitudinal axis of fluid injection system 100. Syringe rod 257 may translate proximally along with syringe body 260 when actuator 250 is unengaged. The proximal translation of syringe rod 257 and/or the proximal translation of syringe body 260 can cause actuator strut 254 to apply force to actuator 250 (e.g., via strut coupling 256 and actuator coupling 252) and cause actuator 250 to assume an unengaged (e.g., un-depressed) configuration, for example, when actuator 250 is unengaged (e.g., as shown in FIG. 3).

Syringe body spring 264 can be held in compression when actuator 250 is not engaged (e.g., when actuator 250 is in an unengaged configuration). For example, syringe body spring 264 can be held in compression between a distal end of syringe body shaft 268 and syringe body 260. In some cases, syringe body 260 is biased against syringe shaft shoulder 267 by syringe body spring 264. In some cases, syringe body shaft 268 and syringe rod shaft 520 are connected interior spaces of fluid injection system 100. In some cases, syringe shaft shoulder 267 denotes a distal end of syringe rod shaft 520 and a proximal end of syringe body shaft 260. The length and/or spring constant of syringe body spring 264 may be designed or selected so that a desired force is required to actuate actuator 250. For example, syringe body spring 264 may be selected to have a length and/or spring constant such that excessive force is not required to actuate actuator 250, which could otherwise decrease a user's control over the position and/or orientation of the device during use or could lead to incomplete actuation of actuator 250. In certain embodiments, it useful to select a syringe body spring 264 to have a length and/or spring constant such that actuator 250 does not actuate under its own weight or if inadvertently bumped, which could lead to unintentional extension of fluid delivery member(s) 320 and/or expression of fluid from fluid delivery member(s) 320.

Low-profile fluid injection system 100 can comprise one or more fluid delivery members 320. Fluid delivery member(s) 320 can comprise a channel through which a fluid can flow. In many cases, fluid injection system 100 comprises a plurality of fluid delivery members 320. For example, fluid injection system 100 can comprise 2, 3, 4, 5, 6, 7, 8, 9, 10, from 10 to 20, from 20 to 30, from 30 to 40, from 40 to 50, or more than 50 fluid delivery members 320. By increasing the number of fluid delivery members 320 comprising fluid injection system 100, more target tissue sites may be injected with fluid. Fluid injection system 100 can inject fluid into a target tissue in well-controlled patterns (e.g., patterns which may comprise one or more column-shaped fluid injection). In some cases, a fluid injection system 100 that comprises a plurality of fluid delivery members 320 will allow multiple different fluids to be injected into one or more portions of a target tissue (e.g., so that effects of each injection may be compared, for example, ex vivo, in situ, in vivo, or in vitro).

Fluid delivery member(s) 320 can comprise a portion of a fluidic pathway (e.g., a continuous fluidic pathway or a valved fluidic pathway) from a source of a fluid (e.g., cartridge 432) to a target tissue (e.g., to an injection site in a target tissue adjacent to or in the vicinity of a distal end of fluid delivery member(s) 320). In some cases, a proximal end 327 of a fluid delivery member 320 is at a greater radial distance from a longitudinal axis of fluid injection system 100 than a distal end 328 of the fluid delivery member 320. A fluid delivery member 320 can comprise one or more bends, which can be advantageous in minimizing the diameter of elongate member (e.g., to reduce the size of an access pathway used to advance elongate member 110 into or through a tissue). In some cases, the number and/or angle of bends in a fluid delivery member 320 depends on the radius at which a proximal end of fluid delivery member 320 is from a longitudinal axis of fluid injection system 100. In some cases, the radius at which a proximal end of fluid delivery member 320 is from a longitudinal axis of fluid injection system 100 depends on the thickness and/or diameter of one or more of: syringe body 260, syringe body spring 264, syringe body shaft 268, syringe rod shaft 520, or cartridge 432. In certain embodiments, the thickness and/or diameter of one or more of: syringe body 260, syringe body spring 264, syringe body shaft 268, syringe rod shaft 520, or cartridge 432 can be minimized to decrease the overall diameter of fluid injection system 100 or to decrease the number or angle of bends in fluid delivery member 320. In some cases, the radius at which a proximal end of fluid delivery member 320 is from a longitudinal axis of fluid injection system 100 depends on the pathway of fluid delivery channel 270 (e.g., the pathway of fluid delivery channel 270 through syringe body 260).

In some cases, one or more fluid delivery members 320 are coupled to syringe body 260, e.g., at a proximal end 327 of fluid delivery members 320. In some cases, one or more fluid delivery members 320 are in fluid communication with one or more fluid delivery channel 270. For example, a proximal end 327 of a fluid delivery member 320 can be in fluid communication with a fluid delivery channel 270, e.g., at delivery channel interface 290.

Fluid delivery channel 270 can be a fluid pathway connecting a fluid source (e.g., cartridge 432) and a fluid delivery member 320. A fluid delivery channel 270 can comprise a portion of one or more of: syringe body 260, cartridge abutment 410, or cartridge interface 420. Low-profile fluid injection system 100 can comprise a plurality of fluid delivery channels 270. For example, low-profile fluid injection system can comprise an equal number of fluid delivery channels 270 as fluid delivery members 320 and/or cartridge chambers 400. In some cases, low-profile fluid injection systems can comprise a plurality of fluidically independent pathways connecting a fluid source (e.g., cartridge 432) to a target tissue. For example, a fluidically independent pathway can comprise a fluid source, a fluid delivery channel 270 and a fluid delivery member 320, wherein the fluidically independent pathway is not in fluid communication with another fluid source (e.g., via a fluid delivery channel 270 and/or a fluid delivery member 320 that is in fluid communication with another fluid source). In some embodiments, fluid injection system 100 comprises 1, 2, 3, 4, 5, 6, 7, 8, 9, 10, from 10 to 20, from 20 to 50, or more than 50 independent fluidic pathways. In some cases, the inclusion of a plurality of fluidically independent pathways in fluid injection system 100 allows for independent treatment and/or subsequent independent analysis of a plurality diagnostic agents and/or a plurality of therapeutic agents.

In some cases, fluid delivery channel 270 or a portion thereof can serve as a fluid reservoir. For example, at least a portion of fluid delivery channel 270 can comprise a fluid to be delivered to a target tissue or portion thereof using fluid injection system 100. In some cases, fluid delivery channel 270 is primed with fluid before fluid injection system 100 is used to inject the fluid into a target tissue or portion thereof. As disclosed further herein, a fluid 480 within a fluid source (e.g., cartridge 432) can be placed under pressure while in fluid communication with fluid delivery channel 270, which can cause fluid 480 to flow from the fluid source into the fluid delivery channel 270.

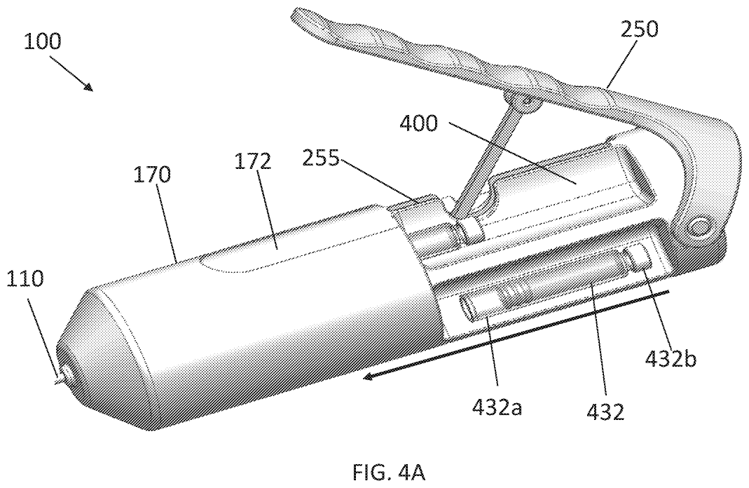

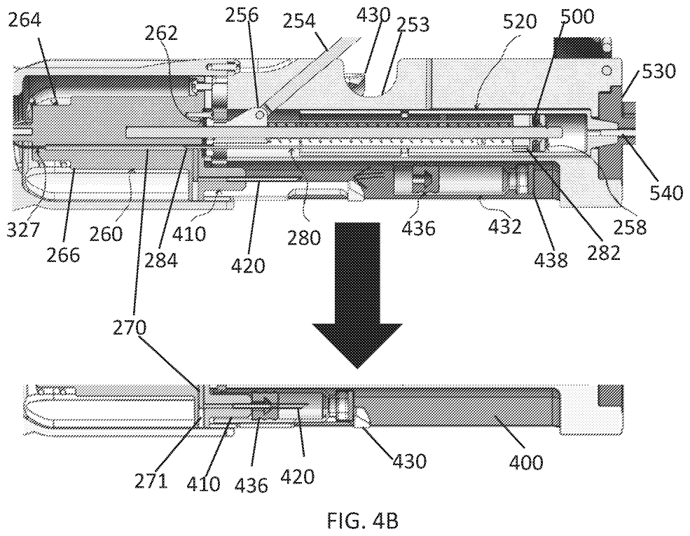

FIG. 4A and FIG. 4B show representative examples of loading cartridge 432 into fluid injection system 100. One or more fluids contained within a cartridge 432 can be placed in fluid communication with fluid delivery channel 270 by engaging cartridge plunger 440 with cartridge interface 420. In some cases, one or more fluids of cartridge 432 can be pressurized when cartridge 432 is loaded into cartridge retainer 430 (e.g., as a result of cartridge 432 being biased against a lip of cartridge retainer 430 by cartridge abutment 410). In some cases, pressurization of a fluid in cartridge 432 during loading of cartridge 432 into fluid injection system 100 can cause the fluid to fill or partially fill fluid delivery channel 270.

In many cases, fluid delivery mechanism 280 (e.g., fluid delivery rod 280) is used to drive fluid from at least a portion of fluid delivery channel 270 toward distal end 114 of a fluid delivery member 320. Fluid delivery mechanism 280 can comprise one or more fluid delivery rods. Fluid delivery rod 280 can be slidably disposed within at least a portion of fluid delivery channel 270. In some cases, fluid delivery rod 280 is sized such that translation of fluid delivery rod 280 down at least a portion of fluid delivery channel 270 (e.g., translation in a distal direction relative to fluid injection system 100) increases pressure inside of at least the portion of fluid delivery channel 270, which may cause expression of fluid from distal end of fluid delivery member 320, e.g., after actuator 250 is actuated. As shown in FIG. 3, fluid delivery rod 280 can be introduced into fluid delivery channel 270 at a bend in fluid delivery channel 270. For example, a distal end 284 of elongate member can be positioned at or adjacent to a bend in fluid delivery channel 270. It is also contemplated that fluid delivery channel 270 may comprise a three-way junction (e.g., a T-junction) wherein fluid delivery rod rests in an arm of the three-way junction in-line with a portion of fluid delivery channel 270 adjacent to and downstream (e.g., distal) of the three-way junction.

Fluid delivery channel 270 can be in fluid communication with a purge channel 271. A purge channel 271 can be in fluid communication with air exterior to fluid injection system 100. In some cases, purge channel 271 comprises a channel and/or a gap through a component of system 100 (e.g., syringe body 260) and or between two or more components of system 100 (e.g., between syringe body 260 and a housing of system 100). In some cases, air (or another gas) present in a channel, reservoir, or cartridge of fluid injection system 100 can be vented through purge channel 271. Purge channel 271 can be useful, e.g. during loading or injection, as excess gases or pressures may be released via purge channel 271.

Fluid injection system 100 can comprise a lockout assembly 500. Lockout assembly 500 can be coupled (e.g., slideably coupled) to syringe rod 257. In some cases, syringe rod 257 is rigidly coupled to syringe rod 257. In some cases, syringe rod 257 can pass through a hole or channel in lockout assembly 500 (e.g., a hole or channel in lockout assembly 500).

Lockout assembly 500 can comprise one or more lockout pins 501. Lockout assembly 500 can comprise 1, 2, 3, 4, 5, 6, 7, 8, 9, 10, or more than 10 lockout pins 501. One or more lockout pins 501 of lockout assembly can be located on a circumferential aspect of lockout assembly 500. For example, one or more lockout pins 501 can protrude from a circumferential aspect of lockout assembly 500. Lockout assembly 500 can comprise one or more springs. In some cases, one or more lockout pins 501 of lockout assembly 500 can be coupled to one or more springs of lockout assembly 500. In some cases, one or more springs of lockout assembly 500 can be configured to bias one or more lockout pins 501 of lockout assembly 500 outwardly (e.g., radially outwardly) from lockout assembly 500. A lockout pin 501 can be configured to anchor lockout assembly 500 at a longitudinal location along syringe rod shaft 520. In some cases, one or more lockout pins 501 of lockout assembly 500 can be biased outwardly against an inner surface of syringe rod shaft 520. In some cases, one or more lockout pins 501 can extend into one or more lockout stops 560 of syringe rod shaft 520 (e.g., as a result of being biased against an inner surface of syringe rod shaft 520 by a spring of lockout assembly 500) to anchor lockout assembly at a longitudinal location of syringe rod shaft 520. In some cases, one or more lockout pins 501 are biased against an inner surface of syringe rod shaft 520 before actuator 250 is engaged and, when actuator 250 is engaged, the one or more lockout pins of 501 slide longitudinally along one or more inner surfaces of syringe rod shaft 520. In cases where syringe rod shaft 520 comprises one or more lockout stops 560 (e.g., along an inner surface of syringe rod shaft 520), one or more lockout pins 501 can be configured to extend at least partially into the one or more lockout stops 560 (e.g., as a result of engaging actuator 250 and/or biasing lockout pins 501 against syringe rod shaft 520 using one or more springs of lockout assembly 500). In some cases, lockout assembly 500 is prevented from translating in a longitudinal direction (e.g., in a distal direction, in a proximal direction, or in both a distal and a proximal direction) when one or more lockout pins 501 are at least partially extended into one or more lockout stops 560.

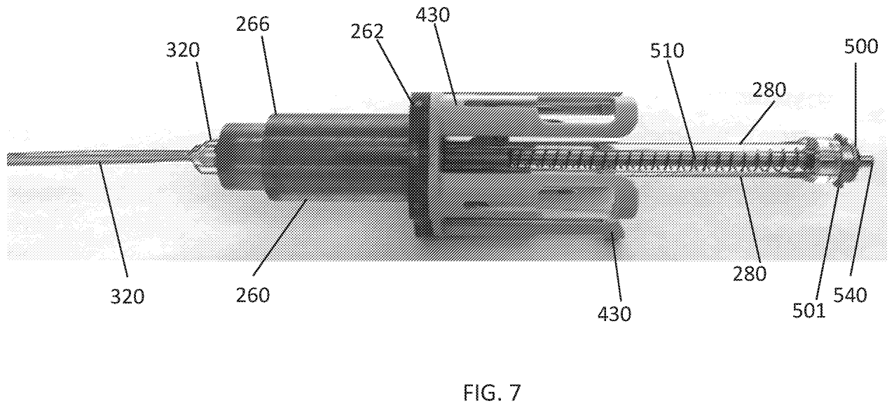

In some cases, such as embodiments wherein fluid injection system 100 is a multiple-use system, one or more lockout pins 501 are configured to be releasably engaged with an aspect of syringe rod shaft 520 (e.g., one or more lockout stops 560). In some cases, one or more lockout pins can be wedge-shaped. For example, a lockout pin can have an angled or beveled surface facing a distal end of fluid injection system 100. In some cases, one or more members, such as a rod or stick, can be introduced into a fluid injection system 100 (e.g., via one or more holes, ports, or channels in a proximal end of fluid injection system 100) to disengage one or more lockout pins 501 from one or more lockout stops 560. A member configured to disengage one or more lockout pins 501 from one or more lockout stops 560 can have a pointed or wedge-shaped distal end. In some cases, forcing one or more members against one or more engaged lockout pins 501 (e.g., via one or more access holes, ports, or channels in a proximal end of system 100) can force the one or more lockout pins 501 back into the body of lockout assembly 500 (e.g., by compressing one or more spring of lockout assembly 500). Disengaging one or more lockout pin 501 from one or more lockout stops 560 can allow one or more components of system 100, such as lockout assembly 500, to travel longitudinally in a proximal direction when actuator 250 is released (e.g., as a result of force applied directly or indirectly to lockout assembly 500 by syringe rod spring 510). FIG. 7 shows an image of internal mechanisms of fluid injection system 100 comprising a lockout assembly 500, lockout pins 501, and syringe rod spring 510.

Fluid injection system 100 can comprise a syringe rod fixture 258 (e.g., a syringe rod pin). Syringe rod fixture 258 may be coupled (e.g., rigidly coupled) to syringe rod 257. In many cases, syringe rod fixture 258 is coupled to syringe rod 257 at a longitudinal location of syringe rod 257 that is proximal to lockout assembly 500 (e.g., relative to a longitudinal axis of fluid injection system 100). In some aspects, syringe rod fixture 258 prevents lockout assembly from sliding off of a proximal end of syringe rod 257 (e.g., due to force exerted by syringe rod spring 510). In some aspects, syringe rod fixture 258 does not inhibit syringe rod 257 from sliding through lockout assembly 500 (e.g., in a proximal direction relative to a longitudinal axis of fluid injection system 100).

Fluid injection system 100 can comprise one or more lockout stops 560. Lockout stops 560 can be fixedly attached to syringe rod shaft 520. In many cases, lockout stops 560 are attached (e.g., fixedly attached) to syringe rod shaft 520 at different locations along a longitudinal axis of fluid injection system 100. In many cases, a plurality of lockout stops 560 is attached to syringe rod shaft 520 at a plurality of locations around the inner circumference of syringe rod shaft 520. In some cases, one or more lockout stops can comprise a continuous spiral shape around syringe rod shaft 520.

In some cases, lockout assembly translates forward (e.g., in a distal direction relative to a longitudinal axis of fluid injection system 100) when syringe rod 257 is translated in a distal direction relative to a longitudinal axis of fluid injection system (e.g., when actuator 250 is engaged). In some cases, lockout assembly 500 can pass lockout stops 560 when lockout assembly translates in a distal direction relative to a longitudinal axis of fluid injection system 100. In many cases, lockout assembly 500 cannot pass one or more lockout stops 560 when lockout assembly translates in a proximal direction relative to a longitudinal axis of fluid injection system 100 (e.g., when actuator 250 is disengaged after being engaged).

In some cases, actuation of volume selector 530 (e.g., rotation of volume selector dial 530) can cause syringe rod shaft 520 and attached lockout stops 560 to rotate within the housing of fluid injection system 100. In some cases, an injection volume is selected by rotating a lockout stop 560 into position such that lockout assembly 500 cannot pass the lockout stop when translating in a proximal direction relative to a longitudinal axis of fluid injection system 100.

Fluid delivery mechanism 280 (e.g., one or more fluid delivery rods 280) can be coupled to lockout assembly 500. In some cases, one or more fluid delivery rods 280 are rigidly attached to lockout assembly 500. In many cases, one or more fluid delivery rods 280 are coupled to lockout assembly 500 at a proximal end 282 of the one or more fluid delivery rods 280. In many cases, when lockout assembly is prevented from translating proximally within the housing of fluid injection system 100 (e.g., relative to a longitudinal axis of fluid injection system 100), fluid delivery rod is also prevented from translating any further in a proximal direction relative to a longitudinal axis of fluid injection system 100 (e.g., when actuator 250 is disengaged after having been engaged).

Fluid injection system 100 can comprise syringe rod spring 510. In some cases, syringe rod spring 510 (e.g., a proximal end of syringe rod spring 510) can be biased against a syringe rod fixture 258 (e.g., a syringe rod pin). In some cases, a syringe rod spring 510 can be biased against a proximal portion of syringe body 260 and/or strut coupling 256. In some cases, syringe rod spring 510 is disposed in compression between syringe rod fixture 258 and one or both of syringe body 260 and strut coupling 256.

In some cases, a spring constant of syringe rod spring 510 is less than that of syringe body spring 264. In some cases, a proximally-directed force acting on syringe body 260 (e.g., as supplied by syringe body spring 264) that is greater than a distally-directed force acting on syringe body 260 (e.g., as supplied by syringe rod spring 510) will allow syringe body 260 to translate in a proximal direction relative to a longitudinal axis of fluid injection system 100, for example, as a result of the unbalanced forces acting on syringe body 260. In some cases, such as various instances when lockout assembly 500 or a portion thereof (e.g., one or more lockout pins 501) has contacted (e.g., engaged) a lockout stop 560, a syringe body spring 264 with a greater spring constant than that of syringe rod spring 510 will cause syringe body 260 and syringe rod 257 to translate proximally while fluid delivery rod(s) 280 and lockout assembly 500 remain in place. In some cases, this can force fluid delivery channel(s) 270 over fluid delivery rod(s) and can result in the propulsion of fluid from at least a portion of fluid delivery channel(s) 270 through fluid delivery member(s) 320 and into the target tissue.

The amount of fluid delivered to a tissue can be related to the distance that one or more fluid delivery members 320 are extended from a distal end 114 of elongate member 110 and/or to the degree to which actuator 250 is engaged (e.g., depressed). In many cases, the amount of fluid that is delivered to a tissue is directly related to the distance that one or more fluid delivery members 320 are extended from a distal end 114 of elongate member 110, which can be directly dependent on the degree to which actuator 250 is engaged. For example, in some cases, an actuator 250 can be depressed further to engage a lockout pin 501 with a lockout stop 560 that is located closer to a distal end of syringe rod shaft 520. In many cases, the act of depressing the actuator 250 further also extends fluid delivery members 320 a further distance from distal end 114 of elongate member 110. Releasing an actuator 250 that has been depressed to a greater degree can also cause fluid delivery mechanism 280 (e.g., fluid delivery rods 280) to push a greater amount of fluid from fluid delivery channel 270, as the syringe body 260 moves proximally within the housing of system 100 (e.g., due to the force exerted against syringe body 260 and an internal portion of the distal end of the housing of system 100 by syringe body spring 264).

### Elongate Members

The system may 100 comprise an elongate member 110 comprising a lumen 112 defined by an inner wall thereof. The lumen 112 may extend the entire length of the elongate member 110, from a proximal end 113 to a distal end 114, along or parallel to a longitudinal axis of the elongate member 110. The elongate member 110 may comprise a hollow tube. For example, the elongate member 110 may comprise a sheath, a hypotube shaft, a needle, or the like. Alternatively, the lumen 112 may extend along any length thereof desired by one of ordinary skill in the art, with any configuration relative to the longitudinal axis of the elongate member 110 desired by one of ordinary skill in the art. The distal end of the lumen 112 may correspond with the distal end 114 of the elongate member 110 as shown.

The elongate member 110 may comprise a metal. The elongate member 110 may comprise stainless steel, nitinol, traditional thermoplastics used in interventional introducers (e.g. HDPE, Pebax, etc.), or the like, or any combination thereof.

The elongate member 110 may comprise a rigid material. Alternatively or in combination, the elongate member 110 may comprise a flexible material.

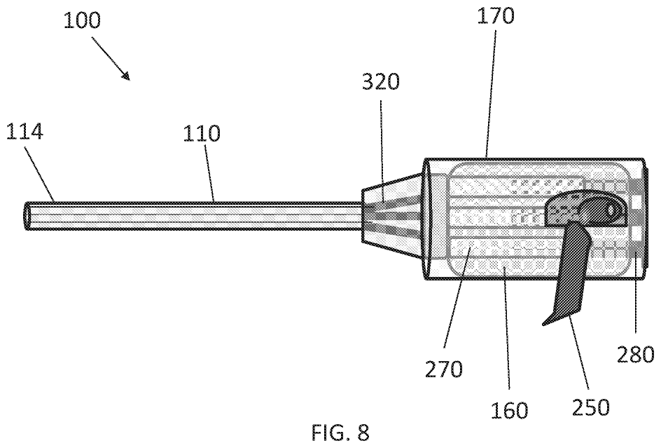

FIG. 8 shows a schematic of a handheld low-profile fluid injection system 100. The system 100 may comprise a plurality of fluid delivery members 320 disposed within an elongate member 110 as described herein. Each of the plurality of fluid delivery members 320 may comprise a fluid delivery lumen therethrough and at least one outlet port 322 at its distal end as described herein. Each of the fluid delivery lumens may be fluidly independent of every other fluid delivery lumen as described herein. The plurality of fluid delivery members 320 may have a retracted configuration and an extended configuration as described herein. The system 100 may comprise one or more fluid delivery channels 270 fluidly coupled to the fluid delivery lumens as described herein. In some embodiments, each fluid delivery channels 270 may be fluidly coupled to a single fluid delivery lumen of the plurality of fluid delivery members 320. For example, a system 100 comprises three fluid delivery members 320 as shown may have three fluid delivery channels 270 fluidly coupled to the fluid delivery members 320 such that each of the fluid delivery member 320 and fluid delivery channels 270 is fluidly independent of every other fluid deliver member 320 and fluid delivery channel 270. The system 100 may comprise one or more fluid delivery channels 280 as described herein. For example, the system 100 may comprise three fluid delivery rods 280 as shown, each of the fluid delivery rods 280 being operably coupled to a single fluid delivery channel (e.g., fluid reservoir) of the three fluid delivery channels (e.g., fluid reservoirs). The three fluid delivery rods 280 may be configured to be operated simultaneously or independently of one another as described herein. Actuation of the fluid delivery rod(s) 280 may cause fluid to be delivered from the plurality of fluid delivery channels 270 to the plurality of fluid delivery members 320 and out of the outlet ports 322 into the tissue of interest. The system 100 may comprise an actuator 250 adjacent to the proximal end of the elongate member 110 and operably coupled to the plurality of fluid delivery members 320 and/or the movable body 160 in order to extend or retract the plurality of fluid delivery members 320 as described herein. The fluid delivery rod 280 may be actuated by the actuator 250 to allow for simultaneous fluid delivery and retraction of the fluid delivery members 320 as described herein.

The housing of system 100 may comprise a handle 170 (e.g., a grip) adjacent to the proximal end of the elongate member 110. In some embodiments, the fluid delivery channels 270 may be located in the handle 170 as shown. In some embodiments, the fluid delivery channels 270 may be located in or coupled to a syringe body 260 slidably disposed within the handle 170 or the elongate member 110.

Alternatively or in combination, the fluid delivery channels 270 may be located external to the handle 170, for example in an external fluid bag fluidly coupled to the proximal end of the handle 170 and/or the fluid delivery members 320 via tubing.

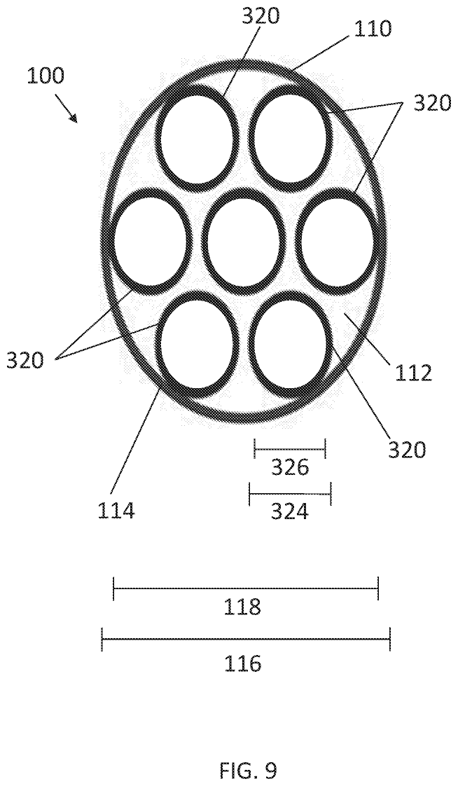

FIG. 9 shows a cross-sectional view of the elongate member 110 of a low-profile fluid injection system 100. A plurality of fluid delivery members 320 may be disposed within the lumen 112 of an elongate member 110 as described herein. The elongate member 110 may have an outer diameter 116 sized for use as a low- or minimally-invasive injection system as described herein. The inner diameter 118 of the elongate member 110, which defines the lumen 112, may determine the size and/or number of fluid delivery members 320 which can be disposed therein. For example, the elongate member 110 may be an 18 G tube having an outer diameter 116 of 1.27 mm and an inner diameter 118 of 0.84 mm. As many as seven 31 G needles having an outer diameter 324 of 0.26 mm may fit within the lumen 112 of the 18 G tube. The plurality of fluid delivery members 320 may have an inner diameter 326 sized to provide fluid delivery as described herein. The size of the elongate member 110 and/or the size of the fluid delivery members 320 may be adjusted as desired in order to provide the system with a desired profile and/or number of fluid delivery members 320.

In some embodiments, the elongate member 110 may comprise a needle, sheath, or tube with a gauge number from about 10 to about 20. The elongate member 110 may for example have an outer diameter 116 in a range bounded by any two of the following gauge numbers: 10, 11, 12, 13, 14, 15, 16, 17, 18, 19, or 20. The elongate member 110 may for example have a gauge number of 10, 11, 12, 13, 14, 15, 16, 17, 18, 19, or 20.

The elongate member 110 may have an outer diameter 116 of from about 0.9 mm to about 3.5 mm. The elongate member 110 may have an outer diameter 116 of from about 2 mm to about 4 mm. The elongate member 110 may have an outer diameter 116 in a range bounded by any two of the following values: 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, or 4 mm.

The elongate member 110 may have an outer diameter 116 of from about 3 French to about 10 French. The elongate member 110 may for example have an outer diameter 116 in a range bounded by any two of the following values: of 3 French, 4 French, 5 French, 6 French, 7 French, 8 French, 9 French, or 10 French. The elongate member 110 may for example have an outer diameter 116 of about 3 French, about 4 French, about 5 French, about 6 French, about 7 French, about 8 French, about 9 French, or about 10 French.

The elongate member 110 may have an outer diameter 116 sized to fit within a working channel of a conventional biopsy access needle, a conventional endoscope, a conventional laparoscopic system, a conventional vascular access sheath, or the like as described herein.

The elongate member 110 may have a longitudinal length of from about 4 cm to about 250 cm. For example, the elongate member 110 may have a length of 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, from 1 cm to 20 cm, from 4 cm to 20 cm, from 5 cm to 15 cm, from 7 cm to 13 cm, or from 9 cm to 11 cm. Alternatively, the elongate member 110 may have a length of from about 100 cm to about 250 cm. The elongate member 110 may for example have a length in a range bounded by any two of the following values: 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 25 cm, 30 cm, 35 cm, 40 cm, 45 cm, 50cm, 75 cm, 100 cm, 125 cm, 150 cm, 175 cm, 200 cm, 225 cm, 250 cm, 275 cm, or 300 cm.

The system 100 may be configured for fluid delivery from about 1 cm to about 300 cm distant from a patient access point (e.g. mouth, skin surface, rectum, etc.). In some embodiments, the system may be configured for fluid delivery from about 1 cm to about 30 cm below the skin surface. For example, the system may be configured for fluid delivery from about 1 cm to about 4 cm below the skin surface or from about 4 cm to about 20 cm below the skin surface. Alternatively, the system may be configured for fluid delivery from about 20 cm to about 40 cm below the skin surface. Alternatively, the system may be configured for fluid delivery from about 100 cm to about 250 cm below the skin surface or from the point on entry into the body (e.g. mouth).

The length of the elongate member 110 used for a particular application may depend on the location of the tissue site of interest. For example, systems 100 with a longer elongate member 110 can be used to delivery one or more agents to a target tissue that is located deeper inside of a subject or a tissue.

### Fluid Delivery Members

One or more fluid delivery members 320 may be disposed within the lumen 112 of the elongate member 110. For example, four fluid delivery members 320 may be sheathed by the elongate member 110 as shown. Any number of fluid delivery members 320 desired may be housed in the lumen 112 of the elongate member 110 as described herein. Each of the fluid delivery members 320 may comprise a distal end, a proximal end, an inner wall defining a fluid delivery lumen therein and an outlet port 322 at its distal end which is fluidly coupled to the lumen. Each of the fluid delivery lumens may be fluidly independent of every other fluid delivery lumen. The one or more fluid delivery members 320 may comprise a plurality of needles or tubes. For example, one or more of the fluid delivery members 320 may comprise a plurality of pencil-point needles, blunt-tipped needles, or bevel-tipped needles.

In some embodiments, each fluid delivery member 320 may comprise a single outlet port 322 at its distal end as described herein. In some embodiments, some or all of the plurality of fluid delivery members 320 may comprise at least one additional outlet port 322 along its exposed length which is fluidly coupled to the fluid delivery lumen, for example as described in PCT/US2008/073212, the entire contents of which are hereby incorporated by reference.

The fluid delivery member(s) 320 may have a retracted configuration and an extended configuration. The fluid delivery members 320 may remain in the retracted configuration while the system 100 is inserted into the body of a patient (e.g. through the skin or mouth 701) and positioned in close proximity to the tumor site 702. The fluid delivery members 320 may be extended out of the distal end 114 of the elongate member 110 to the extended configuration into the tumor 702 as shown in order to deliver the therapeutic agents to the tumor tissue 702. The fluid delivery members 120 may be returned to the retracted configuration for removal of the system 100 from the patient.

The plurality of fluid delivery members 320 may comprise one or more of metal or plastic. The plurality of fluid delivery members 320 may comprise a shape-memory alloy. The plurality of fluid delivery members 320 may comprise stainless steel, nitinol, traditional thermoplastics used in interventional introducers (e.g. HDPE, Pebax, etc.), or the like, or any combination thereof.

The plurality of fluid delivery members 320 may comprise a flexible material. Alternatively or in combination, the plurality of fluid delivery members 320 comprises a rigid material.

In some embodiments, the fluid delivery members 320 may comprise a needle, sheath, or tube with a gauge number in a range of about 28 to about 33. One or more fluid delivery member 320 may be a 25 gauge needle. In some cases, the fluid delivery member 120 may comprise a 20 gauge, 21 gauge, 22 gauge, 23 gauge, 24 gauge, 26 gauge, 27 gauge, 28 gauge, 29 gauge, 30 gauge, 31 gauge, 32 gauge, or 33 gauge needle. The fluid delivery members 320 may for example have an outer diameter 324 in a range bounded by any two of the following gauge numbers: 28, 29, 30, 31, 32, or 33. One or more of the fluid delivery members may for example have a gauge number of 28, 29, 30, 31, 32, or 33.

The fluid delivery members 320 may have an outer diameter 324 in a range of about 0.05 mm to about 0.5 mm. The fluid delivery members 320 may have an outer diameter 324 in a range bounded by any two of the following values: 0.05 mm, 0.06 mm, 0.07 mm, 0.08 mm, 0.09 mm, 0.1 mm, 0.15 mm, 0.2 mm, 0.25 mm, 0.3 mm, 0.35 mm, 0.4 mm, 0.45 mm, or 0.5 mm. One or more of the fluid delivery members 120 may have an outer diameter 124 of about 0.05 mm, about 0.06 mm, about 0.07 mm, about 0.08 mm, about 0.09 mm, about 0.1 mm, about 0.15 mm, about 0.2 mm, about 0.25 mm, about 0.3 mm, about 0.35 mm, about 0.4 mm, about 0.45 mm, or about 0.5 mm.

The system 100 may comprise one or more fluid delivery members 320 disposed within the lumen 112 of the elongate member 110. The system 100 may for example comprise a plurality of fluid delivery members 320. The plurality of fluid delivery members 320 may comprise at least two fluid delivery members 320. The plurality of fluid delivery members 320 may comprise from 2 to 20 fluid delivery members 320. The plurality of fluid delivery members 320 may comprise a number of fluid delivery members 320 in a range bounded by any two of the following values: 2, 3, 4, 5, 6, 7, 8, 9, 10, 11, 12, 13, 14, 15, 16, 17, 18, 19, or 20.

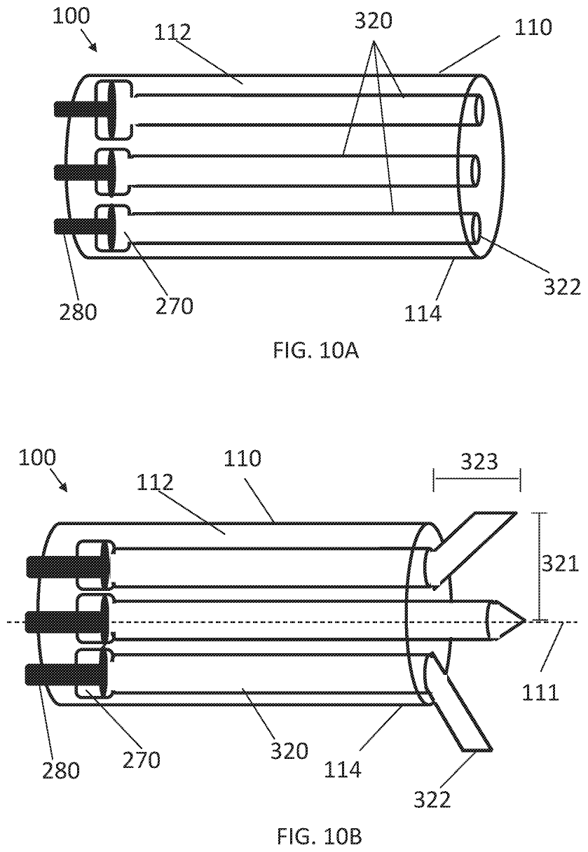

FIG. 10A shows a schematic of a low-profile fluid injection system 100 with fluid delivery members 320 in the retracted configuration. FIG. 10B shows the system 100 with fluid delivery members 320 in the extended configuration. The system 100 may comprise a plurality of fluid delivery members 320 disposed within an elongate member 110 as described herein. Each of the plurality of fluid delivery members 320 may comprise a fluid delivery lumen and at least one outlet port 322 at its distal end as described herein. Each of the fluid delivery lumens may be fluidly independent of every other fluid delivery lumen as described herein. The plurality of fluid delivery members 320 may have a retracted configuration and an extended configuration as described herein.

The fluid delivery members 320 may be configured to be fully enclosed within the lumen 112 of the elongate member 110 in the retracted configuration. In some instances, each of the plurality of fluid delivery members 320 may extend from the distal end 114 of the elongate member 110 to the proximal end of the elongate member 110. For example, the length of each of the plurality of fluid delivery members 320 may be substantially similar to the length of the elongate member 110.

Each of the plurality of fluid delivery members 320 may have a length in a range of about 4 cm to about 250 cm. For example, each of the plurality of fluid delivery members 320 may have a length in a range of about 4 cm to about 20 cm. Alternatively, each of the plurality of fluid delivery members 320 may have a length in a range of about 100 cm to about 250 cm. Each of the plurality of fluid delivery members 320 may for example have a length in a range bounded by any two of the following values: 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 25 cm, 30 cm, 35 cm, 40 cm, 45 cm, 50 cm, 75 cm, 100 cm, 125 cm, 150 cm, 175 cm, 200 cm, 225 cm, 250 cm, 275 cm, or 300 cm.

The length of the fluid delivery members 320 may be adjusted depending on the length of the elongate member 110 and/or the location of the tissue site of interest.

The fluid delivery members 320 may be extended out of the distal end 114 of the elongate member 110 to the extended configuration as described herein. In the extended configuration, each of the plurality of fluid delivery members 320 may angle away from a longitudinal axis 111 of the elongate member 110.

In some embodiments, the distal end 114 of the elongate member 110 may comprise one or more angling elements 115 (e.g., splaying mechanisms) positioned to guide the plurality of fluid delivery members 320 to angle away from a longitudinal axis 111 of the elongate member 110 in the extended configuration. The angling elements or splaying mechanism may for example comprise one or more channels or guides within elongate member 110 that preferentially guide the plurality of fluid delivery members 320 into the desired expanded configuration.

Alternatively or in combination, at least the distal end of each of the plurality of fluid delivery members 320 may comprise a shape memory material or compressible material such that extension of the plurality of fluid delivery members 320 from the distal end 114 of the elongate member 110 allows the distal, exposed end of the each of the plurality of fluid delivery members 320 to self-expand into a separating pattern.

In the extended configuration, each of the plurality of fluid delivery members 320 may angle away from the longitudinal axis 111 of the elongate member 110 at an oblique angle. Each of the plurality of fluid delivery members 320 may angle away from the longitudinal axis 111 of the elongate member 110 at an angle 329 (e.g., a splay angle) in a range of about 10° to about 90°. One or more of the plurality of fluid delivery members 320 may angle away from the longitudinal axis 111 of the elongate member 110 at an angle 329 in a range bounded by any two of the following values: 10°, 15°, 20°, 25°, 30°, 35°, 40°, 45°, 50°, 55°, 60°, 65°, 70°, 75°, 80°, 85°, or 90°. For example, one or more of the plurality of fluid delivery members 120 may angle away from the longitudinal axis 111 of the elongate member 110 at an angle 329 of 10° to 45°, 15° to 30°, or 20° to 25° (e.g., when extended from the distal end 114 of the elongate member 110 outside of a biological tissue or within when extended inside of a biological tissue).

In the extended configuration, each of the plurality of fluid delivery members 320 may angle away from a longitudinal axis 111 of the elongate members 110 such that a distance 321 between distal ends of each of the plurality of fluid delivery members 320 is in a range of about 1 mm to about 10 mm. Each of the plurality of fluid delivery members 320 may angle away from a longitudinal axis 111 of the elongate members 110 such that a distance 321 between distal ends of each of the plurality of fluid delivery members 320 is in a range bounded by any two of the following values: 1 mm, 2 mm, 3 mm, 4, mm, 5 mm, 6 mm, 7 mm, 8 mm, 9 mm, or 10 mm.

In the extended configuration, each of the plurality of fluid delivery members 320 may have a length thereof 323 which extends out of the distal end 114 of the elongate member 110. Length 323 of each of the plurality of fluid delivery members 320 extending out of the distal end 114 of the elongate member 110 in the extended configuration can be in a range of about 1 mm to about 50 mm, for examples in a range of about 5 mm to about 40 mm. Length 323 of each of the plurality of fluid delivery members 320 extending out of the distal end 114 of the elongate member 110 in the extended configuration within may be in a range bounded by any two of the following values: 1 mm, 2 mm, 3 mm, 4 mm, 5 mm, 10 mm, 15 mm, 20 mm, 25 mm, 30 mm, 35 mm, 40 mm, 45 mm, or 50 mm.

### Fluid Delivery Channels

The system 100 may comprise one or more fluid delivery channels 270 (e.g., fluid reservoirs) fluidly coupled to the fluid delivery lumens. In some embodiments, each fluid delivery channel 270 is fluidly coupled to a single fluid delivery lumen of the plurality of fluid delivery members 320. For example, a system 100 comprises three fluid delivery members 320 as shown may have three fluid delivery channels 270 fluidly coupled to the fluid delivery members 320 such that each of the fluid delivery member 320 and fluid delivery channel 270 is fluidly independent of every other fluid deliver member 320 and fluid delivery channel 270. Alternatively, one or more fluid channels 270 or portion thereof may be fluidly coupled to more than one fluid delivery member 320 each. For example, one fluid delivery channel 270 may be fluidly coupled to two fluid delivery members 320. Alternatively or in combination, one or more fluid delivery members 320 may be fluidly coupled to more than one fluid delivery channel 270, for example in the case where mixing of fluids is desired in the fluid delivery member 320. For example, one fluid delivery member 320 may be fluidly coupled to two fluid delivery channels 270 in order to mix two different fluids together in the fluid delivery member 320 during injection.

In some embodiments, the fluid delivery channel(s) 270 may be loaded with fluid(s) prior to inserting the distal end 114 of the elongate member 110 into the body. Alternatively or in combination, the fluid delivery channel(s) 270 may be loaded with fluid(s) during or after inserting the distal end 114 of the elongate member 110 into the body.

In some embodiments, the fluid delivery channels 270 may be directly coupled to a proximal end of the fluid delivery members 320.

In some embodiments, the fluid delivery channels 270 may be fluidly coupled, but not directly coupled, to a proximal end of the fluid delivery members 320.

In some embodiments, the plurality of fluid delivery channels 270 may comprise the fluid delivery lumens of the plurality of fluid delivery members 320. For example, the plurality of fluid delivery channels 270 may be directly and openly coupled to the fluid delivery lumens of the plurality of fluid delivery members 320 such that load the fluids into the plurality of fluid delivery channels 270 also loads (or primes) the fluids into the fluid delivery lumens. In some embodiments, the fluid delivery lumens of the plurality of fluid delivery members 320 may be the plurality of fluid delivery channels 270. That is, the plurality of fluid delivery channels 270 may consist of the fluid delivery lumens of the plurality of fluid delivery members 320 and the fluids may be directly loaded into the fluid delivery lumens.

In some embodiments, the plurality of fluid delivery channels 270 may comprise a plurality of cartridges as described herein.

Each of the plurality of fluid delivery channels 270 may have a volume (i.e. hold a volume of fluid therein) in a range of about 10 μl to about 500 μl. Each of the plurality of fluid delivery channels 270 may for example have a volume in a range bounded by any two of the following values: 10 μl, 20 μl, 30 μl, 40 μl, 50 μl, 60 μl, 70 μl, 80 μl, 90 μl, 100 μl, 125 μl, 150 μl, 175 μl, 200 μl, 225 μl, 250 μl, 275 μl, 300 μl, 325 μl, 350 μl, 375 μl, 400 μl, 425 μl, 450 μl, 475 μl, or 500 μl.

The volume of each fluid delivery channels 270 may comprise the volume of each fluid delivery member lumen fluidly coupled thereto, which may vary depending on the length of the elongate member 110, when the fluid is “primed” in the entire fluid path prior to use.

In some embodiments, the system 100 may comprise one or more label reservoirs (e.g., one or more cartridges 432) fluidly-coupled to one or more of the fluid delivery lumens or one or more of the plurality of fluid delivery channels 270. For example, each fluid delivery member 320 (e.g., each fluid delivery lumen of each fluid delivery member 320) or fluid delivery channel 270 may be fluidly coupled to a label reservoir (e.g., cartridge 432) holding a labeling agent therein. In some instances, each cartridge 432 (e.g., each label reservoir) may be fluidly independent of every other cartridge 432 (e.g., label reservoir). The system 100 may be configured to mix the labeling agent and the therapeutic agent in one or more of the fluid delivery channel 270 or connected fluid delivery lumen such that the fluid injected into the tissue comprises both the labeling agent and the therapeutic agent in the same injection column. In some instances, mixing may occur prior to injection of the therapeutic agent. In some instances, mixing may occur during injection of the therapeutic agent.

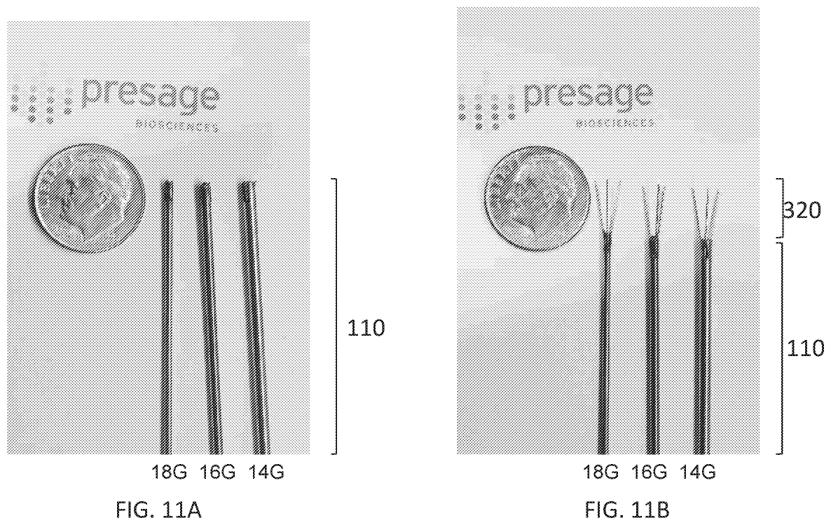

FIG. 11A shows three exemplary prototype low-profile fluid injection systems 100 with three fluid delivery members 320 in the retracted configuration. FIG. 11B shows the systems 100 with fluid delivery members 320 in the extended configuration. The fluid injection systems 100 are shown next to a dime for scale. The three systems 100 were formed using low-profile elongate members 110 having gauge numbers of 18, 16, and 14 (from left to right, respectively, in FIGS. 12A and 12B). In the retracted configuration, the three fluid delivery members 320 were fully enclosed within the elongate member 110 of each system 100. In the extended configuration, the three fluid delivery members 320 splayed out from the distal end 114 of the elongate member 110 at angles away from a longitudinal axis 111 of the elongate member 110 of each system 100 as described herein.

FIG. 12A shows an exemplary low-profile fluid injection system 100 prototype with three fluid delivery members 320 in the retracted configuration. FIG. 12B shows the system 100 with fluid delivery members 320 in the extended configuration. The fluid injection system 100 is shown next to a dime for scale. In the retracted configuration, the three fluid delivery members 320 were fully enclosed within the elongate member 110. In the extended configuration, the three fluid delivery members 320 splayed out from the distal end 114 of the elongate member 110 at angles away from a longitudinal axis 111 of the elongate member 110 as described herein.

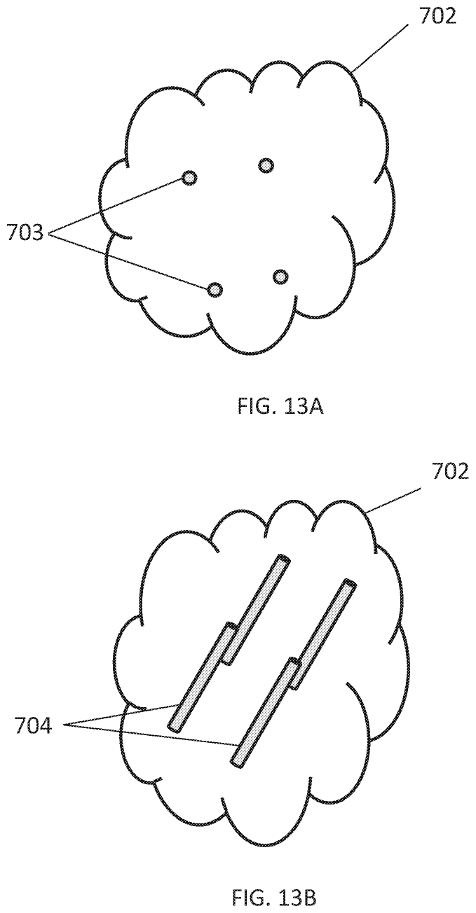

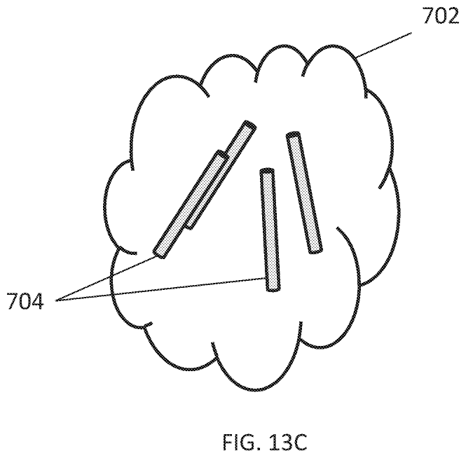

FIG. 13A shows a diagram of a top view of a subdermal tumor tissue 702 following injection with a low-profile fluid injection system 100. FIG. 13B shows a perspective view of injection columns 704 created following injection with a low-profile fluid injection system 100. The system 100 may be configured to inject one or more agents (e.g., drugs) into a tissue at discrete, mapped locations (i.e. injection sites) 703 in order for a user to observe spatially-defined tumor responses to the drugs at the injection sites 703. The agent(s) may be injected into the tissue in a uniform, column-like track 704 through the z-axis of the tissue as shown in FIG. 13B. In some cases, the agent(s) can be injected into a tissue in columns that are parallel to one another. In some cases, the agent(s) can be injected into a tissue in columns that are not parallel to one another (e.g., as shown in FIG. 13C). For example, one or more agents can be injected into a tissue in columns oriented in line with one or more fluid delivery members (e.g., when the fluid delivery members are in an extended (e.g., splayed) configuration). One or more agents may be injected into the tumor with a label as described herein in order to aid in identification of the drug candidates and/or confirm successful drug delivery.

The system 100 may be configured to inject a plurality of fluids at a plurality of injection sites 703 to form a plurality of injection columns 704 within the tissue. In some embodiments, each of the fluid delivery members 320 may inject a different fluid/agent, such that the number of distinct agents/fluids injected into the tumor is the same as the number of fluid delivery members 320/injection sites 703. In other embodiments, one or more fluid delivery member 320 may inject the same fluid/agent, such that the number of distinct agents/fluids injected into the tumor is less than the number of fluid delivery members 320/injection sites 703. Alternatively or in combination, one or more of the fluid delivery members 320 may inject fluids/agents having the same active ingredient but at different concentrations.

The drugs may be left in the tumor 702 for a pre-determined period of time before resection and analysis, for example about 24 hours to about 72 hours. During that time, the drugs may diffuse into the tissue immediately surrounding the injection columns 704. The injection columns 704 produced by the system 100 may be spaced in such a way so as to prevent cross-contamination or to allow the drugs to mix within the tissue, as desired by one of ordinary skill in the art.

The tissue 702 may be resected for analysis of the therapeutic efficacy and/or toxicity of the drugs. Assessing the therapeutic efficacy of the drug may for example include analyzing the tissue 702 for known markers of cytotoxicity, hypoxia, angiogenesis, immune response, dysregulation of a target biochemical or genetic pathway, or the like, or any combination thereof.

The tissue 702 may be sampled at multiple tumor depths in order to assess the consistency of the tumor response to drug, which may be of particular use for very heterogeneous tumor types with spatially-varying microenvironments. The resected tissue may for example be cut into a plurality of serial sections at a predetermined interval along the injection column and analyzed by any known histological, histochemical, immunohistological, immunohistochemical, histopathologic, microscopic, cytological, biochemical, pharmacological, molecular biological, immunochemical, imaging, or other analytical technique, or combination thereof, known to one of ordinary skill in the art.

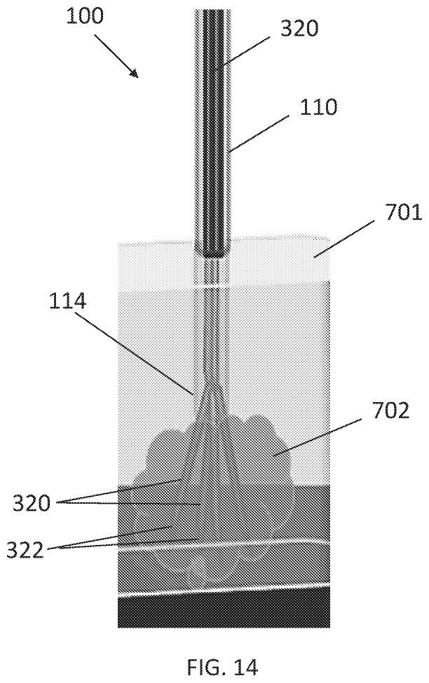

FIG. 14 shows a schematic of a low-profile fluid injection system 100. The system 100 may be used to deliver one or more agents, for example therapeutic agents or drugs, through the skin 701 or other access point (e.g. the mouth), to an internal target tissue 702, for example a subdermal tumor.

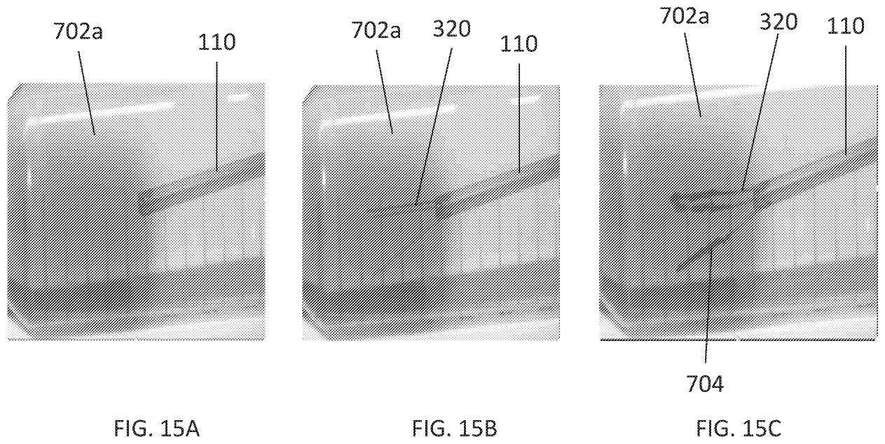

FIG. 15A shows a distal end of an exemplary low-profile fluid injection system 100 comprising an angling element and three fluid delivery members 320 in an unextended (e.g., retracted) configuration adjacent to a simulated tumor tissue 702a. Simulated tumor tissue 702a comprised a 0.55% agarose gel dyed with red food coloring inside of a test tube. The distal end 114 of the elongate member 110 was positioned adjacent to simulated tumor tissue 702a. The three fluid delivery members 320 were then extended into the simulated tumor tissue 702a. As shown in FIG. 15B, fluid delivery members 320 were successfully extended into the simulated tumor tissue at oblique angles. As disclosed herein, actuator 250 can be engaged to cause fluid delivery members 320 to assume an extended configuration, as shown in FIG. 15B. FIG. 15C shows the system 100 during fluid injection into simulated tumor tissue 702a and simultaneous retraction of the fluid delivery members 320. The fluid delivery members 320 were retracted at a rate of 0.75 mm/s while 1 microliter of each fluid, comprising 50% green food coloring, was injected into the simulated tumor tissue 702a. Simultaneous injection and retraction resulted in clear injection columns 702 within simulated tumor tissue 702a.

### Fluid Delivery Mechanisms

The system 100 may comprise one or more fluid delivery mechanism 280. In many cases, a fluid delivery mechanism can comprise a fluid delivery rod 280. A fluid delivery mechanism can comprise a plurality of fluid delivery rods 280. Actuation of the fluid delivery rod(s) 280 may cause fluid to be delivered from the plurality of fluid delivery channels 270 to the plurality of fluid delivery members 320 and out of the outlet ports 322 into the tissue of interest.

In some embodiments, fluid delivery mechanism 280 may comprise a single fluid delivery rod 280 operably coupled to each of the plurality of fluid delivery channels 270 such that actuation of the fluid delivery rod 280 causes fluid to be delivered from each of the plurality of fluid delivery members 320 at the same time.

Alternatively, fluid delivery mechanism 280 may comprise a plurality of fluid delivery rods. In some embodiments, each of the plurality of fluid delivery rod 280 may be operably coupled to a single fluid delivery channel 270 (e.g., fluid reservoir) of the plurality of fluid delivery channels 270 (e.g., fluid reservoirs). In some embodiments, the plurality of fluid delivery rod 280 may function independently of one another such that each of the plurality of fluid delivery members 320 may deliver fluid independently of every other fluid delivery member 320. In some embodiments, each of the plurality of fluid delivery rod 280 may be operably coupled to more than one fluid delivery channel 270 (e.g., one fluid reservoir) of the plurality of fluid delivery channels (e.g., fluid reservoirs).

The fluid delivery mechanism 280 may comprise a mechanical actuator or an electromechanical actuator. In some embodiments, the fluid delivery rod 280 may comprise one or more of a plunger or a pump. In some embodiments, a fluid delivery rod 280 comprises a gasket (e.g., a rubber gasket or a plastic gasket). For example, a fluid delivery rod 280 can comprise a gasket at its distal end, which can be configured to form a water-tight junction with an inner aspect (e.g., an inner wall surface) of a fluid delivery channel. In some cases, a fluid delivery rod 280 does not comprise a gasket. In many embodiments, a fluid delivery rod 280 is configured to slide through a fluid channel (e.g., a fluid delivery channel) or reservoir. Moving a fluid delivery rod 280 within a fluid channel or reservoir (e.g., sliding a fluid delivery rod 280 through a fluid delivery channel or reservoir) can cause a fluid within the fluid delivery channel or reservoir to move. For example, moving a fluid delivery rod 280 distally relative to fluid delivery channel or reservoir can cause a fluid within the fluid delivery channel or reservoir to move distally within the fluid delivery channel or reservoir. In some cases, moving a fluid delivery channel or reservoir proximally relative to a fluid delivery rod 280 can cause a fluid within the fluid delivery channel or reservoir to move distally relative to the fluid delivery channel or reservoir. A diameter of a fluid delivery rod 280 can be sized relative to an inner diameter of a fluid delivery channel 270 or reservoir so that a fluid in the fluid delivery channel 270 or reservoir is moved when fluid delivery rod 280 is moved relative to the fluid delivery channel 270 or reservoir.

The fluid delivery rod 280 may be manually operated. Alternatively or in combination, the fluid delivery rod 280 may be automatically operated, for example by a computer program as described herein.

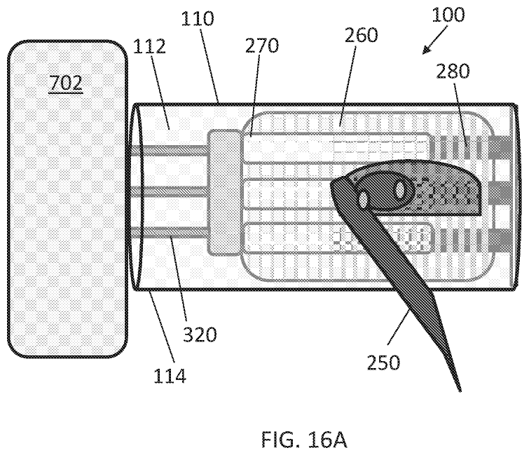

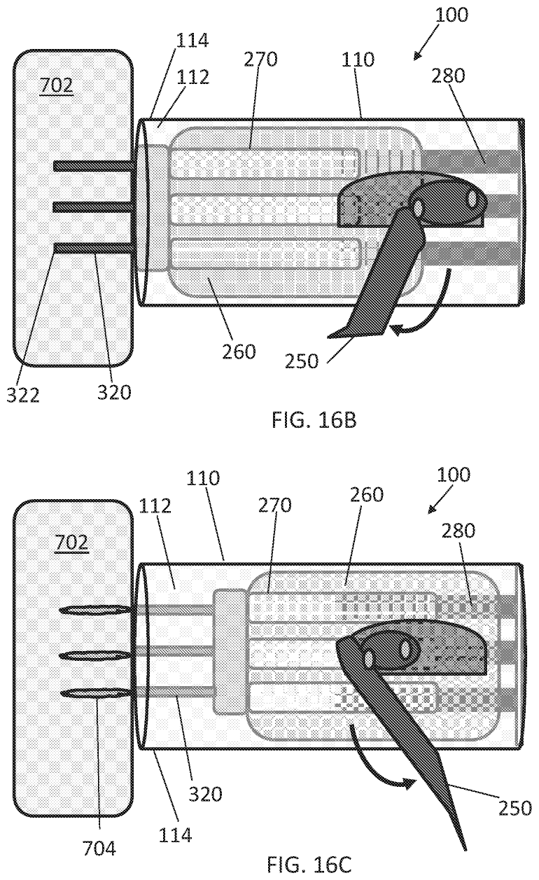

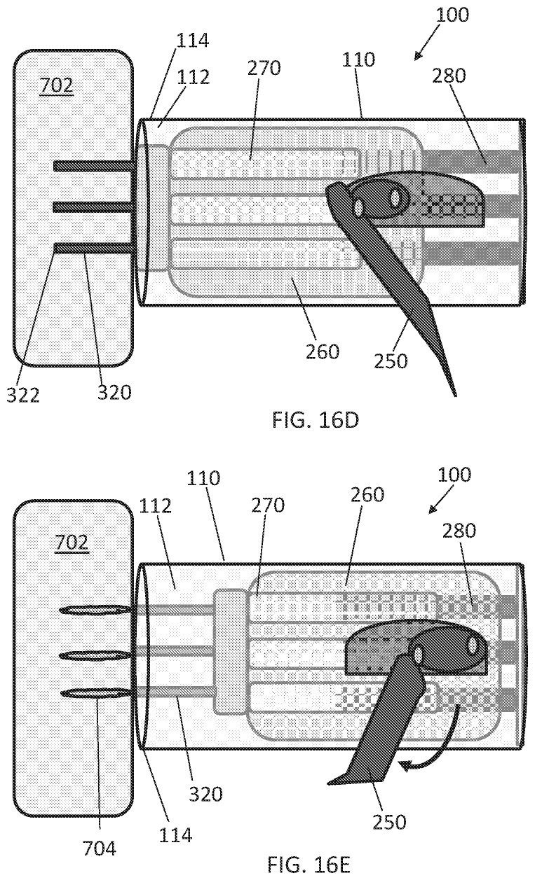

FIG. 16A shows a schematic of a low-profile fluid injection system 100 prior to fluid injection with fluid delivery members 320 in an unextended configuration. In some cases, actuator 250 can be engaged to extend one or more fluid delivery members 320 into a tissue 702 (e.g., as shown in FIG. 16B). In some cases, the extent to which actuator 250 is engaged determines the distance that fluid delivery members 320 are extended into tissue 702. Fluid delivery members 320 can retract into the fluid injection system (e.g., into elongate member 110), as shown in FIG. 16C. In some cases, retraction of fluid delivery members 320 is passive (e.g., due to the action of a spring internal to system 100) or active (e.g., as a result of pulling actuator 250 back to its initial position).

FIG. 16D shows a schematic of a low-profile fluid injection system 100 prior to fluid injection with fluid delivery members 320 in an extended configuration. FIG. 16E shows the system 100 after simultaneous fluid injection and retraction of the fluid delivery members 320. The system 100 may comprise a plurality of fluid delivery members 320 disposed within an elongate member 110 as described herein. Each of the plurality of fluid delivery members 320 may comprise a fluid delivery lumen therethrough and at least one outlet port 322 at its distal end as described herein. Each of the fluid delivery lumens may be fluidly independent of every other fluid delivery lumen as described herein. The plurality of fluid delivery members 320 may have a retracted configuration and an extended configuration as described herein. The system 100 may comprise one or more fluid delivery channels 270 fluidly coupled to the fluid delivery lumens as described herein. In some embodiments, each fluid delivery channel 270 may be fluidly coupled to a single fluid delivery lumen of the plurality of fluid delivery members 120. For example, a system 100 comprises three fluid delivery members 320 as shown may have three fluid delivery channels 270 fluidly coupled to the fluid delivery members 320 such that each of the fluid delivery member 320 and fluid delivery channel 270 is fluidly independent of every other fluid deliver member 320 and fluid delivery channel 270. The system 100 may comprise one or more fluid delivery rod 280 as described herein. For example, the system 100 may comprise three fluid delivery rod 280 as shown, each of the fluid delivery rod 280 being operably coupled to a single fluid delivery channel 270 (e.g., fluid reservoir 130) of the three of fluid delivery channels 270 (e.g., fluid reservoirs). The three fluid delivery rods 280 may be configured to be operated simultaneously or independently of one another as described herein. Actuation of fluid delivery rod 280 may cause fluid to be delivered from the plurality of fluid delivery channels 270 to the plurality of fluid delivery members 320 and out of the outlet ports 322 into the tissue of interest.

### Actuators

The system 100 may comprise an actuator 250 (e.g., expansion actuator 250) adjacent to the proximal end of the elongate member 110 and operably coupled to the plurality of fluid delivery members 320 and/or a syringe body 260 operably coupled thereto as described herein. Actuator 250 can remain at an angle 259 relative to a longitudinal axis 101 of fluid injection system 100 when the actuator is unengaged. In certain embodiments, actuator angle 259 can be from 10 degrees to 180 degrees, from 10 degrees to 90 degrees, from 30 degrees to 90 degrees, from 30 degrees to 60 degrees, or from 30 degrees to 45 degrees. In some cases, actuator angle 259 can be an angle around actuator hinge 251. In some cases, actuator angle 259 can be measured relative to a plane parallel to a longitudinal axis 101 of fluid injection system 100. For example, an actuator angle 259 can be measured relative to a plane parallel to a longitudinal axis 101 that runs through actuator hinge 251.

Actuation of actuator 250 may move the plurality of fluid delivery members 320 from the retracted configuration to the expanded configuration or from the expanded configuration to the retracted configuration. The actuator 250 may comprise a mechanical actuator or an electromechanical actuator.

Actuator 250 may be manually operated. Alternatively or in combination, actuator 250 may be automatically operated, for example by a computer program as described herein.

In some embodiments, the fluid delivery rod 280 may be actuated by the actuator 250 to allow for simultaneous fluid delivery and retraction of the fluid delivery members 320 as described herein. Alternatively or in combination, the fluid delivery rod(s) 280 may be actuated independently of the actuator 250.

Actuation of the fluid delivery rod(s) 280 may be operably coupled to the plurality of fluid delivery members 320 and/or a syringe body 260 of the system 100 such that delivery of fluid is concomitant with retraction of the fluid delivery members 320 from the extended configuration to the retracted configuration. The plurality of fluid delivery members 320 may be configured to retract from the extended configuration to the retracted configuration simultaneously with fluid delivery from the fluid delivery members 320. Simultaneous fluid delivery and retraction of the fluid delivery members 320 may aid in the formation of clean injection columns 704 in the tissue of interest (as shown in FIG. 9).

Simultaneous fluid delivery and retraction of the fluid delivery members 320 may be achieved by “pulling” the fluid delivery channels 270 and fluid delivery members 320 towards the stationary fluid delivery rod(s) 280 within the body of the system 100. The fluid delivery channels 270 may for example be operably coupled to or located within a syringe body 260 of the system 100 slidably disposed within the elongate member 110 or a handle or the like. Retraction of the fluid delivery members 320 from the extended configuration (shown in FIG. 16D) to the retracted configuration (shown in FIG. 9) may comprise retracting the syringe body 260, and the fluid delivery channels 270 located therein, from a distal position to a proximal position in order to engage the stationary fluid delivery rod(s) 280 and cause fluid to flow from the fluid delivery channels 270 to the distal end of the fluid delivery members 320 and out of the outlet ports 322. This mechanism of action may be in contrast to traditional plunger-syringe-like mechanisms where the fluid delivery rod(s) are “pushed” into stationary fluid delivery reservoirs (e.g., fluid delivery channels 270) located in the body of the system.

Simultaneous fluid delivery or retraction of the fluid delivery members 320 may be achieved by electromechanical means. For example, coordinated gears may retract the fluid delivery members 320 while micropumps may inject fluid from the fluid delivery members 320.

The actuator 250 may be configured to retract the plurality of fluid delivery members 320 from the expanded configuration to the retracted configuration at the same speed. Alternatively, the actuator 250 may be configured to retract one or more of the plurality of fluid delivery members 320 at different speeds, for example in order to maintain the same fluid delivery volume per area for fluids of differing viscosities or flow rates.

The fluid delivery members 320 may be retracted at a speed sufficient to generate an injection column 704 as described herein.

The fluid delivery members 320 may be retracted at a speed in a range of about 0.1 mm/s to about 10 mm/s. For example, the speed may be in a range bounded by any two of the following values: about 0.1 mm/s, about 0.2 mm/s, about 0.3 mm/s, about 0.5 mm/s, about 1 mm/s, about 2 mm/s, about 3 mm/s, about 4 mm/s, about 5 mm/s, about 6 mm/s, about 7 mm/s, about 8 mm/s, about 9 mm/s, or about 10 mm/s.

Each of the fluid delivery channels 270 may hold the same volume of fluid. Alternatively, one or more of the fluid delivery channels 270 may hold different volumes of fluid.

Each of the plurality of fluid channels 270 may have a volume in a range of about 10 μl to about 500 μl. For example, the volume of a fluid channel 270 may be in a range bounded by any two of the following values: about 10 μl, about 20 μl, about 30 μl, about 40 μl, about 50 μl, about 75 μl, about 100 μl, about 150 μl, about 200 μl, about 250 μl, about 300 μl, about 350 μl, about 400 μl, about 450 μl, or about 500 μl.

Each of the fluid delivery lumens of the plurality of fluid delivery members 320 may hold the same volume of fluid. Alternatively, one or more of the fluid delivery lumens of the plurality of fluid delivery members 320 may hold different volumes of fluid.

Each of the fluid delivery lumens of the plurality of fluid delivery members 320 may have a volume in a range of about 0.1 μl to about 10 μl. For example, the volume of a fluid delivery member lumen may be in a range bounded by any two of the following values: about 0.1 μl, about 0.2 μl, about 0.3 μl, about 0.5 μl, about 1 μl, about 2 μl, about 3 μl, about 4 μl, about 5 μl/s, about 6 μl, about 7 μl, about 8 μl, about 9 μl, or about 10 μl.

The volume of each of the fluid delivery lumens may depend on the length of its corresponding fluid delivery member 320, which may be varied depending on the length of the elongate tube 110 and the location of the tissue site of interest.

The fluid delivery rod 280 may be configured to cause fluid to be delivered out of the outlet ports 322 at a flow rate sufficient to generate an injection column 704 as described herein with minimal generation of shear forces and induction of mechanochemical damage to the tissue 702.

The fluid delivery rod 280 may be configured to cause fluid to be delivered out of the outlet ports 322 at a flow rate in a range of about 0.1 μl/s to about 10 μl/s. For example, the flow rate may be in a range bounded by any two of the following values: about 0.1 μl/s, about 0.2 μl/s, about 0.3 μl/s, about 0.5 μl/s, about 1 μl/s, about 2 μl/s, about 3 μl/s, about 4 μl/s, about 5 μl/s, about 6 μl/s, about 7 μl/s, about 8 μl/s, about 9 μl/s, or about 10 μl/s.

### Volume Selectors

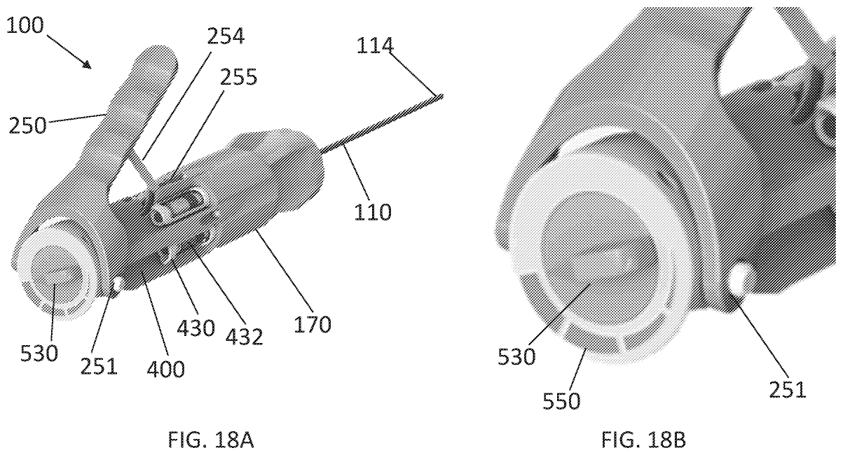

FIG. 18A and FIG. 18B shows a fluid injection system 100 comprising volume selector 530. In many cases, volume selector 530 is used to control the volume of fluid injected into a target tissue. Volume selector 530 can be disposed at a proximal end of fluid injection system 100. Volume selector 530 can be coupled (e.g., rigidly coupled) to volume adjustment screw 540. Volume adjustment screw 540 can be coupled to syringe rod shaft 520. In some cases, actuating (e.g., rotating) volume selector 530 can actuate syringe rod shaft 520 (e.g., rotate syringe rod shaft 520 about a longitudinal axis 101 of fluid injection system 100). In some cases, volume selector 530 comprises a dial. In some cases, volume selector 530 can be used to set a volume to be injected into a target tissue by rotating the dial to a selected volume position. In some cases, volume selector 530 can be used to select a volume to be injected from a plurality of discrete volumes. In some cases, volume selector can be used to select a volume from a continuous range of volumes. IN some embodiments, volume selector 530 can be used to set a volume for injection of 1 microliter to 1.5 microliters, 1.5 microliters to 2.0 microliters, 2.0 microliters to 2.5 microliters, 2.5 microliters to 3.0 microliters, 3.0 microliters to 3.5 microliters, 3.5 microliters to 4.0 microliters, 4.0 microliters to 4.5 microliters, 4.5 microliters to 5.0 microliters, 5.0 microliters to 5.5 microliters, 5.5 microliters to 6.0 microliters, 6.0 microliters to 6.5 microliters, 6.5 microliters to 7.0 microliters, 7.0 microliters to 7.5 microliters, 7.5 microliters to 8.0 microliters, 8.0 microliters to 8.5 microliters, 8.5 microliters to 9.0 microliters, 9.0 microliters to 9.5 microliters, 9.5 microliters to 10.0 microliters, 10.0 microliters to 50.0 microliters, 50.0 microliters to 100.0 microliters, 100.0 microliters to 500.0 microliters, or more than 500.0 microliters. In some cases, actuating volume selector 530 may actuate one or more lockout stops of fluid injection system 100. In some cases, actuating one or more lockout stops of fluid injection system 100 can comprise rotating syringe rod shaft 520 (e.g., wherein rotating syringe rod shaft 520 comprises rotating one or more lockout stops 560 into a position to engage lockout assembly 500). As disclosed herein, a selected volume can be related to the distance that one or more fluid delivery members 320 extend from a distal end 114 of elongate member 110 when actuator 250 is engaged. For example, selecting a larger volume for delivery using volume selector 530 can increase the distance that one or more fluid delivery members 320 extend from distal end 114 of elongate member 110 when actuator 250 is engaged. Volume selector 530 can comprise one or more volume setting indicator 550. Volume setting indicator 550 can comprise one or more visual and/or tactile features. In some cases, the one or more visual and/or tactile features can comprise information regarding possible injection volume settings.

### Distal Caps

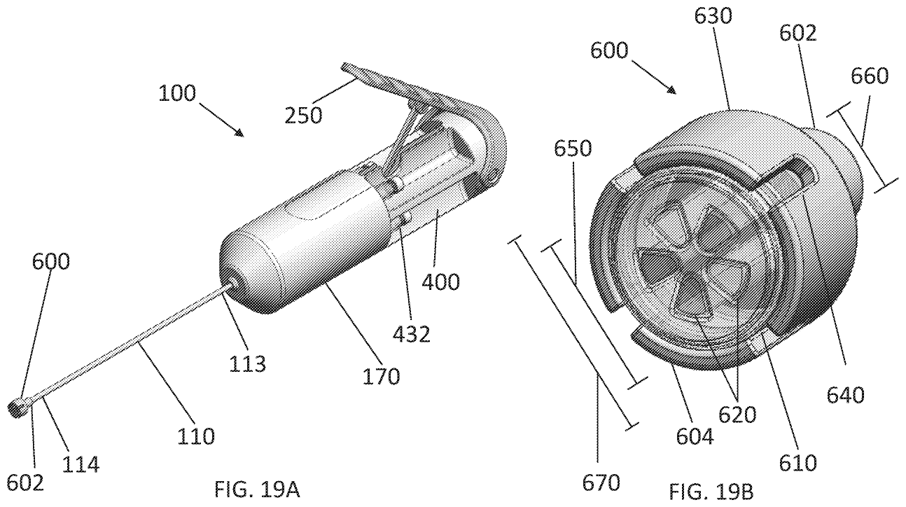

Turning to FIG. 19A and FIG. 19B, fluid injection system 100 can comprise a distal cap 600. A distal cap can be useful in preventing accidental leakage of a fluid (e.g., an agent) comprised by fluid injection system 100. One or more fluids to be delivered to a target tissue may be hazardous if allowed to contact non-target tissues (e.g., the skin of the subject or the skin of a bystander). In some cases, distal cap 600 can prevent accidental contact of one or more fluids of fluid injection system 100 with a non-target tissue. Distal cap 600 can comprise one or more cap reservoirs 620. In some cases, a cap reservoir 620 of distal cap 600 can be useful in collecting fluids from the distal end(s) 328 of one or more fluid delivery members 320. A distal cap 600 can also be helpful in determining whether one or more channels, apertures (e.g., openings), or reservoirs of system 100 is clogged and would impair fluid flow and can be helpful in ensuring that one or more channels and/or reservoirs of fluid injection system 100 is fully filled (e.g., to guard against incomplete filling). For example, one or more fluid delivery members 320 of fluid injection system 100 can be dipped into a fluid comprising one or more agents contained in one or more insert reservoirs 630 of distal cap 600 to fill one or more channels and/or reservoirs of fluid injection system 100. Loading fluid injection system 100 from a distal cap 600 can also be useful in reducing the volume of liquid and/or solid agents to be delivered to (e.g., injected into) a tissue.

Distal cap 600 can comprise a cap insert 610. Cap insert 610 can comprise one or more cap reservoirs 620. In some cases, cap insert 610 comprises 1, 2, 3, 4, 5, 6, 7, 8, 9, 10, from 10 to 20, from 20 to 30, from 30 to 40, from 40 to 50, or more than 50 cap reservoirs 620. A cap reservoir 620 of cap insert 610 can have a fluid capacity of 0.1 microliter to 10 microliters, 10 microliters to 20 microliters, 20 microliters to 50 microliters, 50 microliters to 100 microliters, 100 microliters to 200 microliters, 200 microliters to 500 microliters, 500 microliters to 1000 microliters, or more than 1000 microliters.

Distal cap 600 can comprise an insert receiver 630. In various embodiments, cap insert 610 and insert receiver are configured such that cap insert may be fitted inside of insert receiver 630, e.g., while cap insert 610 and insert receiver 630 are each seated on a distal end 114 of elongate member 110. In some cases, the inner diameter of a distal portion of insert receiver 630 is the same as the outer diameter 650 of a distal portion of cap insert 610. In some cases, the inner diameter of a distal portion of insert receiver 630 is from 0.1 mm to 0.2 mm, from 0.2 mm to 0.5 mm, from 0.5 mm to 1.0 mm, from 1.0 mm to 5.0 mm, or more than 5.0 mm larger than the outer diameter 650 of a distal portion of cap insert 610. Distal end of insert receiver 630 can have an outer diameter 670 of 0.5 mm to 1.0 mm, 1.0 mm to 2.0 mm, 2.0 mm to 3.0 mm, 3.0 mm to 4.0 mm, 4.0 mm to 5.0 mm, 5.0 mm to 6.0 mm, from 6.0 mm to 7.0 mm, or larger than 7.0 mm.

Insert receiver 630 can comprise one or more receiver notches 640. Receiver notch 640 can be a cutout feature of insert receiver 630, e.g., located on a distal edge of insert receiver 630. In some cases, receiver notch 640 can be useful in removing cap insert 610 from insert receiver 630. For example, a cap insert 610 with a distal end that is flush with the distal end of insert receiver 630 can be removed from insert receiver 630 by contacting cap insert 610 in the space created by receiver notch 640 and guiding cap insert 610 out of insert receiver 630. In some cases, cap insert 610 can be removed from distal end 114 of elongate member 110 without removing insert receiver 630 from distal end 114 (e.g., if a used cap insert is being changed out for a different cap insert).

Distal cap can comprise a proximal end 602 and a distal end 604. In some cases, a proximal end 602 of distal cap 600 is shaped to receive distal end 114 of elongate member 100. Proximal end 602 of distal cap 600 can have an inner diameter 660 equal to or slightly larger than the outer diameter of distal end 114 of elongate member 110. In some cases, distal end 114 of elongate member 110 has a structural feature configured to retain distal cap on the distal end of elongate member 110. For example, distal end 114 of elongate member can comprise an indentation shaped to mate with a lip or fastening mechanism on the proximal end 602 of distal cap 600.

Distal cap 600 can be used to fill (e.g., to load) at least a portion of fluid injection system 100 with one or more fluids comprising one or more agents.

### Cartridges

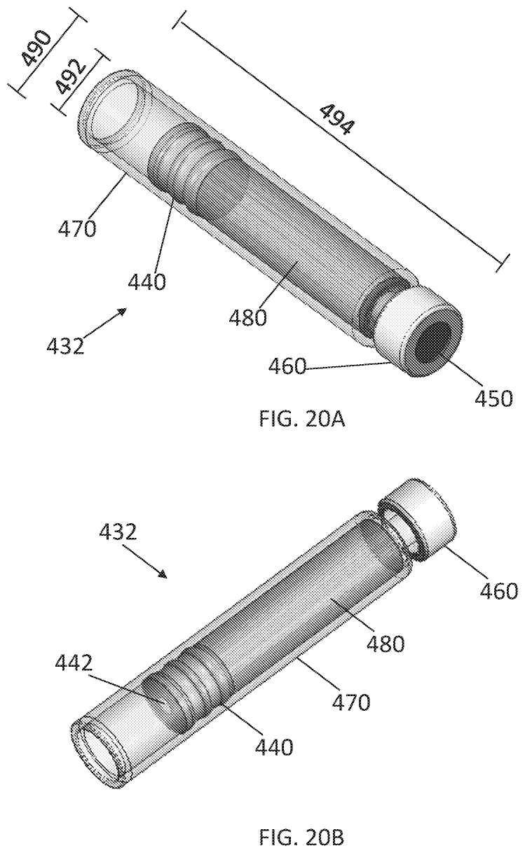

Turning to FIG. 20A and FIG. 20B, low-profile fluid injection system 100 may comprise a cartridge 432. Cartridge 132 may be removable from fluid injection system 100. Cartridge 432 can comprise a cartridge shell 470. In some cases, cartridge 132 is disposable. In some cases, cartridge 432 is reusable. Cartridge 432 or one or more portions thereof can be autoclavable. Fluid injection systems 100 that comprise a removable and/or reusable cartridge 432 can have improved versatility. For example, one or more cartridges 432 to be used with fluid injection system 100 can be prepared beforehand. In some cases, one or more cartridges 432 can be prepared remotely and transported or shipped to a location at which they will be used. Low-profile fluid injection systems 100 comprising one or more cartridges 432 can also be reconfigured from a first injection configuration to a second injection configuration easily (e.g., by substituting and/or rearranging one or more cartridges 432 used in the fluid injection system).

The cartridge(s) 432 may be fully or partially filled with fluid(s) prior to being inserted or loaded into chamber 400. A cartridge 432 may be pre-filled by a technician or pharmacist before the cartridge 432 is loaded into a chamber of device 100. In some cases, a pre-filled cartridge 432 can be stored, shipped, or frozen. Alternatively or in combination, the cartridge(s) 432 may be loaded with fluid(s) after being inserted or loaded into the chamber 400. For example, the cartridge(s) 432 may be loaded with a fluorescent label prior to being inserted into the chamber 400 and subsequently loaded with a drug compound after being inserted into the chamber 400.

A cartridge may be pre-loaded with one or more agents (e.g., one or more therapeutic agents, one or more indicators, and/or one or more buffers or excipients). In some embodiments, the cartridge(s) 432 may be pre-loaded with one or more indicators (e.g., labels). Alternatively or in combination, the cartridge(s) 432 may be pre-loaded with one or more therapeutic compounds.

Cartridge 432 can comprise a cartridge stopper 450. Cartridge stopper 450 can comprise one or more of various materials useful for capping a vial. Cartridge stopper can comprise a polymer or a copolymer. Cartridge stopper 450 can comprise a natural rubber or a synthetic rubber (e.g., butyl rubber). In some cases, cartridge stopper 450 comprises a self-healing material (e.g., a material capable of maintaining a water-tight seal after being punctured). In some cases, cartridge 432 can be loaded by injecting one or more fluids (e.g., one or more agents) through cartridge stopper 450.

In some cases, cartridge 432 further comprises a stopper seal 460. In some cases, stopper seal 460 is configured to hold cartridge stopper 450 in place at an end (e.g., a proximal end 432b) of cartridge 432 and/or to aid in maintaining a water-tight seal at an end of cartridge 432 (e.g., by exerting a compressive force on cartridge stopper in an end of cartridge 432). Stopper seal 460 can comprise a metal, polymer, co-polymer, or ceramic material. In some cases, stopper seal 460 is a crimp seal.

A cartridge 432 can comprise cartridge plunger 440. In some cases, cartridge plunger 440 is slidably inserted or positioned at a longitudinal position inside cartridge 432. For example, cartridge plunger can be positioned at a longitudinal position inside cartridge 432 that is closer to distal end 432a of cartridge 432 than proximal end 432b of cartridge 432. In some cases, cartridge plunger 440 is translated distally down a longitudinal axis of cartridge 432 when cartridge 432 is loaded with one or more fluids (e.g., one or more agents), for example, by injecting the one or more fluids through cartridge stopper 450. Cartridge plunger 440 can comprise plunger interface 442. In some cases, plunger interface 442 can comprise a material puncturable by a needle (e.g., a self-healing material). In some cases, plunger interface 442 can comprise a mechanism configured to place the contents of cartridge 432 into fluid communication with fluid delivery channel 270, such as a port configured to engage with delivery channel interface 290.

Cartridge 432 can comprise one or more cartridge reservoirs configured to hold a volume of a fluid 480. In some cases, cartridge 432 comprises a plurality of cartridge reservoirs. In some cases, two or more cartridge reservoirs of a plurality of cartridge reservoirs of cartridge 432 can be in fluid communication with one another. For example, a pressure applied to cartridge plunger 440 (e.g., when cartridge 432 is loaded into chamber 400 of fluid injection system 100) can cause the contents of two or more cartridge reservoirs of cartridge 432 to mix.

Various cartridges 432 disclosed herein may be configured to hold a volume of fluid 480 (e.g., by slideably inserting cartridge plunger 440). Cartridge 432 can be configured to hold a specific volume of fluid by changing the position of cartridge plunger 440. In some cases, cartridge 432 is configured to hold a volume of 1 microliter to 500 microliters, 10 microliters to 500 microliters, 100 microliters to 500 microliters, 200 microliters to 500 microliters, 300 microliters to 500 microliters, 1 microliter to 250 microliters, 1 microliter to 100 microliters, 1 microliter to 50 microliters, 1 microliter to 40 microliters, 1 microliter to 30 microliters, 1 microliter to 20 microliters, 1 microliter to 10 microliters, 1 microliter to 9 microliters, 1.25 microliters to 9 microliters, 2 microliters to 8 microliters, 3 microliters to 7 microliters, 3.75 microliters to 6.5 microliters, 4 microliters to 6 microliters, or 0.1 microliters to 1 microliter. The volume of a cartridge 432 may be in a range bounded by any two of the following values: about 1 μl, about 2 μl, about 3 μl, about 4 μl, about 5 μl, about 6 μl, about 7 μl, about 8 μl, about 9 μl, or about 10 μl. Each of a plurality of cartridges 432 of fluid injection system 100 may hold the same volume of fluid. Alternatively, one or more of the plurality of cartridges 432 may hold different volumes of fluid.

Cartridge 432 can have an outer diameter of 2.0 mm to 3.0 mm, 3.0 mm to 4.0 mm, 4.0 mm to 5.0 mm, 5.0 mm to 6.0 mm, from 6.0 mm to 7.0 mm, from 7.0 mm to 8.0 mm, from 8.0 mm to 9.0 mm, from 9.0 mm to 10.0 mm, or larger than 10.0 mm. Cartridge 432 can have an inner diameter of less than 1.0 mm, 1.0 mm to 2.0 mm, 2.0 mm to 3.0 mm, 3.0 mm to 4.0 mm, 4.0 mm to 5.0 mm, 5.0 mm to 6.0 mm, 6.0 mm to 7.0 mm, 7.0 mm to 8.0 mm, 8.0 mm to 9.0 mm, 9.0 mm to 10.0 mm, or larger than 10.0 mm.

Cartridge 432 may be configured to be inserted into a correspondingly-shaped recess or chamber 400in the housing of fluid injection system 100. In some cases, cartridge 432 can be held in position by cartridge retainer 430 after being loaded into chamber 400 of fluid injection system 100. Cartridge retainer 430 can comprise various structural elements for holding cartridge 432 in position (e.g., during use of fluid injection system 100). For example, cartridge retainer 430 can comprise a spring mechanism for biasing cartridge 432 against chamber 400 and/or against cartridge abutment 410. In many cases, cartridge retainer 430 comprises a clip for holding cartridge 432 in position in chamber 400. Cartridge retainer 430 can comprise a lip shaped to fit over a proximal end 432b of cartridge 432 when cartridge 432 is pressed against cartridge abutment 410. A representative example of a cartridge retainer 430 comprising a lip is shown in FIG. 4B.

In some cases, positioning cartridge 432 in chamber 400 (e.g., by engaging cartridge 432 with cartridge retainer 430) can cause cartridge abutment 410 to apply a compressive force to cartridge plunger 440. In some cases, a force applied to cartridge plunger 440 (e.g., by cartridge abutment 410) can cause pressurization of fluid 480 inside of cartridge 432. In some cases, pressurization of fluid 480 inside of cartridge 432 can cause fluid 480 to flow from cartridge 432 into fluid delivery channel 270 (e.g., via cartridge interface 420). In some embodiments, the cartridge(s) 432 may be directly coupled to a proximal end of a fluid delivery member 320 (e.g., when cartridge 432 is engaged with cartridge retainer 430.

In some embodiments, the cartridge(s) 432 may be loaded with fluid(s) prior to inserting the distal end 114 of the elongate member 110 into the body. Alternatively or in combination, the cartridge(s) 432 may be loaded with fluid(s) during or after inserting the distal end 114 of the elongate member 110 into the body.

In some embodiments, the cartridge(s) 432 may be loaded into the system 100 prior to inserting the distal end 114 of the elongate member 110 into the body. Alternatively or in combination, the cartridge(s) 432 may be loaded into the system 100 during or after inserting the distal end 114 of the elongate member 110 into the body.

In some embodiments, one or more of the cartridges 432 may comprise a label reservoir as described herein. The one or more cartridges 432 may for example hold a labeling agent therein. The one or more cartridges 432 may be configured to mix the labeling agent and the therapeutic agent therein such that the fluid injected into the tissue comprises both the labeling agent and the therapeutic agent in the same injection column. In some instances, mixing may occur prior to injection of the therapeutic agent. In some instances, mixing may occur during injection of the therapeutic agent.

### Agents

A fluid 480 of cartridge 432 can comprise one or more agents. One or more agents of fluid 480 can be a therapeutic agent. For example, fluid 480 can comprise one or more drugs, such as an antitumor drug. In some cases, fluid 480 comprises a plurality of agents. In some cases, fluid 480 comprises a plurality of therapeutic agents. In many cases, two cartridges 432 of fluid injection system 100, comprise different agents or different combinations of agents.

In some cases, one or more agents of fluid 480 can be a diagnostic agent. For example, fluid 480 can comprise an indicator agent. An indicator agent can comprise a fluorescent dye, a chromophoric dye, or a fiduciary marker.

One or more agents of fluid 480 can comprise a fluorescent tracking molecule. A fluorescent tracking molecule can be helpful in tracking the region(s) of a target tissue contacted by a fluid 480 injected into the target tissue (e.g., using fluid injection system 100). Importantly, the use of fluorescent tracking molecule in fluid injection system 100 can aid in determining a relative location and/or orientation of a target tissue, e.g., during a second time point or after explantation of the target tissue.

A fluorescent tracking molecule can be a microparticle. A fluorescent tracking molecule can be a fluorescent tracking microsphere (FTM). For example, a fluorescent tracking molecule can be a polymer microsphere. A fluorescent tracking molecule can comprise polystyrene. In some cases, polystyrene can provide performance advantages during data acquisition and analysis steps. For example, polystyrene is resistant to harsh chemicals that may be used during imaging and analysis of injected tissue, such as xylenes, which are commonly used in histological processes and can adversely affect certain polymers and/or leach dye from an indicator molecule comprising various other materials. In some cases, tissue processing can be performed with aliphatic hydrocarbons (e.g., Clear-Rite™ 3) to improve dye retention of FTM particles. A cross-linker can be used to enhance chemical heat resistance properties of an FTM particle. For example, an FTM particle can comprise DVB-crosslinked polystyrene. A DVB crosslinker can be used at a concentration from 0.1% to 5% during FTM particle formation. In some cases, a fluorescent tracking microsphere (FTM) can comprise benzoguanamine formaldehyde resin. In some cases, fluorescent tracking microspheres can offer the advantage of allowing for microspheres to be sectioned using common sectioning practices. In such situations, it can be less likely that the microspheres will be dragged across the tissue in which they are injected during the process of sectioning, which can cause tearing of the tissue and/or displacement of the particles relative to one another or the tissue.

Advantages of fluid injection systems 100 comprising fluorescent tracking microspheres (FTM) include the ability to track one or more agents and/or fluids delivered to a tissue can be precisely and to process a tissue containing one or more FTM without damaging the tissue or causing significant negative effect on the brightness of the FTM. Additionally, FTM retain excellent brightness and visibility in a tissue, even when the FTM are formulated with relatively small amounts of dye.

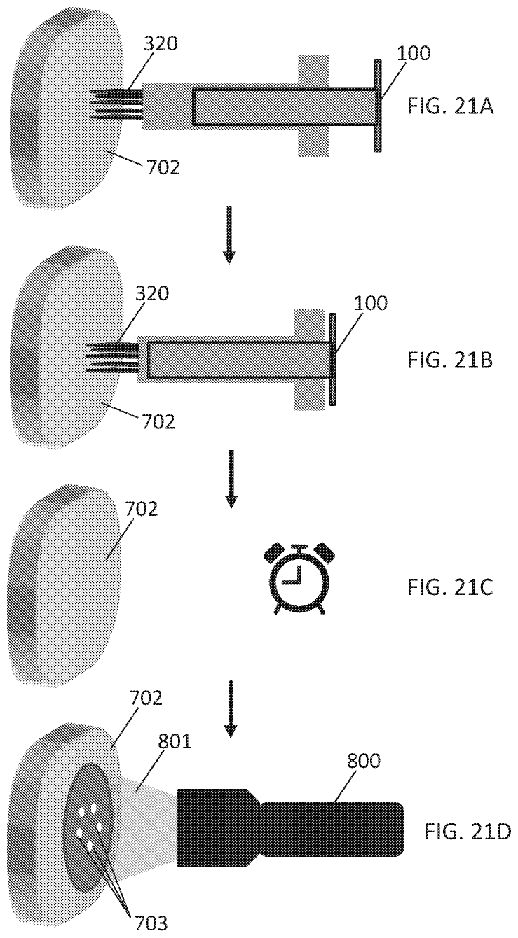

FTM can be delivered to a plurality of sites 703 in a tissue 702 using a fluid injection system, such as a system 100 disclosed herein, wherein one or more FTM are detected in the tissue 702 prior to resection of the tissue (e.g., as shown in FIGS. 21A-21D). By using a radiation source 800, such as a visible light source or an ultraviolet light source (e.g., in the form of a handheld device), it is possible to determine a location, an orientation, and/or one or more boundaries of one or more injection sites 703 in a tissue, even if the injected tissue has been moved or an injection site has healed. In many cases, the use of FTM in such situations is superior to both the use of metal fiducial implants in conjunction with imaging methods (e.g., fluoroscopy, ultrasound, or computer tomography) and the use of tattoos at least because FTM can be imaged readily using a handheld radiation source, because FTM do not require specialized detectors (e.g., they can often be identified visually when imaged), and because FTM are compatible with assays (e.g., immunohistochemistry, fluorescent imaging with or without the use of antibodies, or in situ hybridization) performed on tissue after delivery of the tracking particles (e.g., after resection of the tissue). Accordingly, the use of FTM particles can reduce or eliminate the need for large, expensive imaging equipment, for example, because FTM particles can be imaged and evaluated quickly and intuitively using a smaller (e.g., handheld) radiation source, such as a handheld UV light. Lights and filters used in illumination and detection of injection site can be compact and handheld allowing for quick and economical detection as compared to large fluoroscopic equipment used in surgical settings to detect metal fiducial markers placed within tumors in biopsy & resection processes.

Other examples of injection devices, systems and methods that can be used with FTM include those disclosed in U.S. Pat. Nos. 8,349,554, 8,657,786, 8,834,428, 8,475,412, 8,672,887, 8,926,567, 9,205,201, and 9,205,202, which are incorporated herein in their entireties for all purposes. Methods of using FTM disclosed herein can also be applied to other devices, systems, and methods, such as those disclosed in U.S. Pat. Nos. 8,349,554, 8,657,786, 8,834,428, 8,475,412, 8,672,887, 8,926,567, 9,205,201, and 9,205,202, which are incorporated herein in their entireties for all purposes.

It can be advantageous to control the size of fluorescent tracking microspheres (FTM) delivered to a tissue. For example, particles greater than 100 nanometers in diameter resist movement after injection, which may be due to changes in local fluid pressures, diffusion, and/or deformation of the tissue. Particles having a diameter 5 micrometers or smaller can be less likely to be phagocytosed by a cell in an injected tissue. A fluorescent tracking molecule can be from 0.1 micrometers to 1.0 micrometers, 1.0 micrometers to 5.0 micrometers, 5.0 micrometers to 10.0 micrometers, 4.0 micrometers to 11.0 micrometers, 4.0 micrometers to 12.0 micrometers, or 1.0 micrometers to 20.0 micrometers. In many cases, a plurality of FTM particles to be delivered to a tissue (e.g., loaded into a cartridge or distal cap or located within a fluid delivery channel or reservoir of system 100) can have diameter within a C.V. range of from 0.1% to 1.0%, from 1.0% to 2.0%, from 2.0% to 3.0%, from 3.0% to 4.0%, from 4.0% to 5.0%, or from 5.0% to 10.0%. In some cases, a first cartridge 432 or fluid delivery channel 270 can comprise an FTM population having a first diameter, and a second cartridge or fluid delivery channel 270 can comprise a second FTM population having a second diameter. In some cases, a first set of one or more agents delivered to a tissue with a first population of FTM can be differentiated from a second set of one or more agents delivered to the tissue with a second population of FTM by the relative or absolute sizes (e.g., diameter) and/or the signals (e.g., emitted fluorescent wavelength) of the first and second FTM populations. Accordingly, it is possible to create many distinctly identifiable FTM populations using a relatively small number of fluorescent dyes, which can require fewer fluorescence imaging channels of a detector to distinguish. For example, using two diameters of FTM particles and two different dyes, it is possible to create six uniquely identifiable FTM particle populations (e.g., using either dye alone or using the two dyes together). Fluorescent tracking microspheres (e.g. sized from 5.0 micrometers to 10.0 micrometers) are small enough to travel with a fluid injected into a target tissue, small enough that they are not likely to be phagocytosed, and large enough that they are likely to remain localized during tissue analysis and, if applicable, processing.

A fluorescent tracking microsphere (FTM) can comprise a dye. For example, an FTM particle can comprise a polymer microsphere stained with one or more dyes. A fluorescent tracking molecule can comprise an organic dye. An organic dye of a fluorescent tracking molecule can be a fluorescent organic dye. In some cases, an aqueous dye, such as an aqueous UV dye, can be used in an FTM particle, however, organic dyes are superior in many applications since they are less prone to leaching out of a microparticle in an aqueous environment. FTM can be formulated to comprise a dye (e.g., a fluorescent dye) in a range from 0.1% to 0.4%, 0.01% to 1%, or 0.1% to 5% (% weight to weight) weight content per bead. In some cases, a fluorescent tracking molecule can have an excitation wavelength from 450 nm to 495 nm or from 300 nm to 600 nm. Dyes that can be used with FTM (e.g., incorporated into FTM particles) can comprise Nile Red, Yellow 160, BODIPY dyes, Lucifer yellow, xanthene derivatives (e.g., fluorescein, fluorescein isothiocyanate (FITC), rhodamine, tetramethylrhodamine (TRITC), Oregon green, eosin, Texas red), cyanine derivatives (e.g., Cy2, Cy3, Cy3B, Cy3.5, Cy5, Cy5.5, Cy7, cyanine indocarbocyanine, oxacarbocyanine, thiacarbocyanine, merocyanine), squaraine derivatives, squaraine rotaxane derivatives, naphthalene derivatives, coumarin derivatives, oxadiazole derivatives (e.g., pyridyloxazole, nitrobenzoxadiazole, benzoxadiazole), anthracene derivatives, pyrene derivatives (e.g., cascade blue), oxazine derivatives (such as Nile red, Nile blue, cresyl violet, oxazine 170), acridine derivatives, arylmethine derivatives, or tetrapyrrole derivatives. In some cases, each cartridge 432 comprising fluid injection system 100 can have a different detection wavelength or range of detection wavelengths. A dye of an FTM particle can produce a detectable signal (e.g., upon excitation by a source of radiation, such as a visible light lamp or a UV light). In some cases, a signal from an FTM particle can be detected using one or more detectors. In some cases, a signal from an FTM particle is visually assessed (e.g., by a surgeon, technician, nurse, histologist, researcher, or other scientist). A signal from an FTM particle (e.g., from a dye of an FTM particle can be from 350 nm to 750 nm, from 400 nm to 600 nm, from 450 nm to 550 nm, from 400 nm to 500 nm, from 500 nm to 600 nm, greater than 750 nm, or less than 350 nm.

In many cases, a fluid injection system 100 can be loaded with FTM particles comprising different dyes or combinations of dyes. For example, a first fluid delivery member can be loaded with and/or used to deliver a first FTM population comprising a different set of one or more dyes than a second population of FTM particles loaded into or delivered using a second fluid delivery member. Accordingly, a first set of one or more agents delivered to a tissue from a first fluid delivery member can be differentiated from a second set of one or more agents delivered to a tissue from a second delivery member.

Due to the intense brightness of fluorescent tracking microspheres, FTM can be added to a drug for delivery to a tissue at a concentration of from 0.1% to 5%, 5% to 10%, 10%, 10% to 20%, 20% to 30%, 30% to 40%, 40% to 50%, or greater than 50%. In some cases, FTM (e.g., polystyrene FTM) can be formulated (e.g., with one or more agents) in a fluid to be delivered to a tissue at 35 milligrams/milliliter (mg/ml) to 45 mg/ml, 25 mg/ml to 50 mg/ml, 15 mg/ml to 60 mg/ml, 10 mg/ml to 65 mg/ml, 0.01 mg/ml to 1 mg/ml, 1 mg/ml to 10 mg/ml, or greater than 60 mg/ml. In some cases, formulation of FTM in a fluid for delivery in a range of 10 mg/ml to 50 mg/ml provides the best brightness and density of FTM particles.

One or more agents of fluid injection system 100 can be an implantable agent. For example, one or more agents comprising system 100 or delivered to a tissue using system 100 can be an implantable agent, such as an implant configured for controlled-release of a substance. An implantable agent can comprise a pellet, a powder, a slurry, or a microdevice. In some cases, an implantable agent can comprise an injectable micropump. In some cases, an implantable agent can comprise a degradable matrix, such as a degradable polymer matrix. An implantable agent can be configured to deliver (e.g., release) one or more agents (e.g., drugs) into a tissue during and/or after injection. In some cases, one agent can be delivered to a tissue by an implantable agent. In some cases, a plurality of agents can be delivered to a tissue by an implantable agent. An implantable agent delivered by system 100 can comprise a bioabsorbable material.

An implantable agent (e.g., a degradable polymer particle or micropump) can be configured to release one or more agents into a tissue at a constant rate or at a variable rate. In some cases, the rate of release of one or more agents into a tissue by an implantable agent can increase over time. In some cases, the rate of release of one or more agents into a tissue by an implantable agent can decrease over time. In some cases, the rate of release of one or more agents into a tissue by an implantable agent can both increase and decrease In some cases, control of the rate of release of an agent into a tissue by an implantable agent can be accomplished by engineering a degradable particle to have greater or lower amounts of the agent at different locations within the implantable agent and/or by selection of the composition of the implantable agent (e.g., by selecting the type or ratio of one or more polymers or co-polymers comprising the various portions of the implantable agent) and/or varied distribution of the agent to be delivered through the implantable agent. The use of implantable agents can be advantageous for controlling exposure of a tissue to one or more agents.

In some cases, an implantable agent can be a fiducial marker, e.g., for marking a position in a tissue. For example, an implantable agent can comprise one or more pellets or pill-shaped implants that can be delivered through one or more fluid delivery members 320 to a tissue of a subject for marking an injection location. In some cases, an implantable agent comprising a fiducial marker can comprise a metal or a metal alloy. In some cases, an implantable agent comprising a fiducial marker can be detectable with an electromagnetic field and/or using a radiation source or visual inspection.

### Applications

The devices, systems, and methods described herein may be used for delivery of any agent to a solid tissue for therapeutic or non-therapeutic purposes.

The devices, systems, and methods described herein may be used for pre-clinical drug development and testing and/or clinical drug development and testing.

The devices, systems, and methods described herein may be used for personalized medicine applications, for example to determine the most efficacious therapeutic agent, or combination of agents, for an individual patient's tumor treatment.

The devices, systems, and methods described herein may be used to access, and deliver one or more fluids to, a target site within the body. The target site may for example be at a location from about 1 cm to about 300 cm distant from the patient access point (e.g. mouth, skin surface, rectum, etc.). The target site may for example be at a location from about 1 cm to about 30 cm below the skin surface. The target site may, for example, be at a location 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, from 1 cm to 20 cm, from 5 cm to 15 cm, from 7 cm to 13 cm, or from 9 cm to 11 cm below the skin surface. The target site may for example be at a location from about 4 cm to about 20 cm below the skin surface. The target site may for example be at a location from about 100 cm to about 250 cm distant from a patient access site.

The target site may for example be a superficial target site which may be accessed transcutaneously, for example at a location from about 0.2 cm to about 4 cm deep in a human patient.

The target site may for example be an intermediate target site which may be accessed transcutaneously, for example at a location from about 4 cm to about 20 cm deep in a human patient.

The target site may for example be a deeper target site which may be accessed endoscopically or interventionally, for example at a location from about 100 cm to about 250 cm deep from the point of entry (e.g. from the mouth to the stomach) in a human patient.

In some instances, the target site may be a tumor. The tumor may be located anywhere within the body of a patient. The tumor may for example be located in the skin, breast, brain, prostate, colon, rectum, kidney, pancreas, lung, liver, heart, stomach, intestines, ovaries, testes, cervix, lymph nodes, thyroid, esophagus, head or neck, eye, bone, or bladder of the patient. The tumor may be located in any location within the body where solid tumors are found.

Tumors that can be treated with the devices, systems, and methods described herein include, but are not limited to, gastric carcinomas, esophageal cancers, liver metastases from colon carcinoma, papillary renal carcinomas, head and neck cancers, thyroid cancers, ovarian cancers, cervical cancers, lymphomas, skin cancers (e.g. melanomas, etc.), pancreatic cancers, prostate cancers, testicular cancers, renal-cell carcinomas, breast cancers, colorectal cancers, brain cancers (e.g. medulloblastomas, glioblastomas, etc.), lung cancers (e.g. mesothelioma, small cell lung cancer, non-small cell lung cancer, etc.), liver cancers (e.g. hepatocellular carcinomas, etc.), bladder cancers, rhabdomyosarcomas, and osteosarcomas.

The devices, systems, and methods described herein may be used to deliver one or more therapeutic agents to a tissue of interest. The therapeutic agent(s) may be delivered in a liquid form. Exemplary therapeutic agents for cancer treatment include, but are not limited to, general chemotherapeutics, bisphosphonates, hormone therapies, antibodies, immunotherapies (e.g. CAR T-cells, NK cells, etc.), steroids, angiogenesis inhibitors, proteasome/protease inhibitors, tyrosine kinase inhibitors, interferons, interleukins, and the like, and any combination thereof.

The devices, systems, and methods described herein may be used to deliver one or more labels (also referred to herein as tags or probes). The label(s) may be delivered with another agent, for example a therapeutic agent, or as a single agent. The label(s) may be conjugated to another agent, for example a therapeutic agent, or delivered with another agent in solution (unbound). The label(s) may aid in the detection of the injection sites or columns using conventional imaging techniques as described herein and known to one or ordinary skill in the art. Exemplary labels include, but are not limited to, fluorescent labels, radiolabels, gas chromatography/mass spectrometry (GCMS) tags, chemically-inert visible injection tracking dyes (ITDs), and the like, and combinations thereof.

### Methods

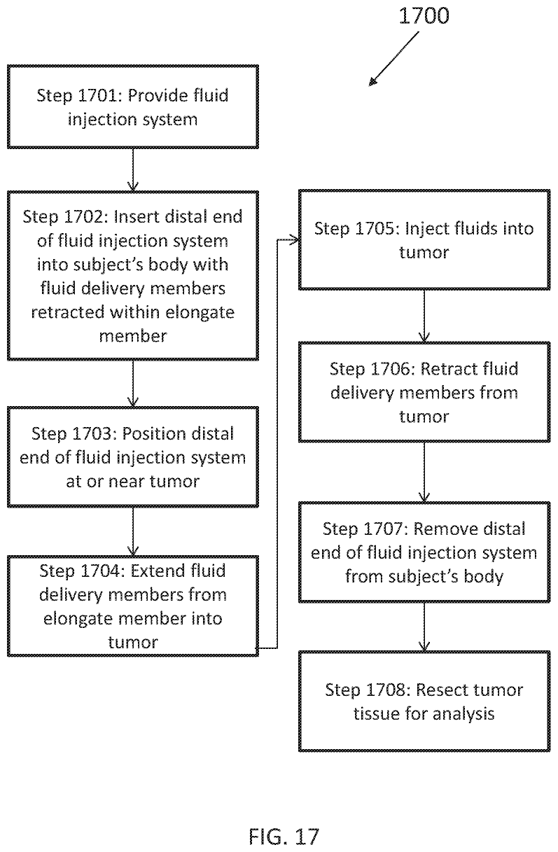

FIG. 17 shows a method 1700 of injecting fluid into a tumor within a body of a patient using a fluid injection system 100 as described herein. The method may use one or more of the systems and apparatus described herein.

At step 1701, a fluid injection system may be provided. The fluid injection system may be any of the fluid injection systems 100 described herein. The fluid injection system may for example comprise an elongate member, a plurality of fluid delivery members disposed therein, and a plurality of fluid reservoirs (e.g., fluid delivery channels) fluidly coupled to the plurality of fluid delivery members. Each of the plurality of fluid reservoirs (e.g., fluid delivery channels) may be coupled to a single fluid delivery member, with each of the fluid delivery members being fluidly independent from every other fluid delivery member.

Providing a fluid injection system 100 (e.g., as in step 1701) can comprise providing a cartridge 432, such as those disclosed herein. The cartridge 432 can be loaded into the fluid injection system 100. For example, cartridge 432 can be loaded into fluid injection system 100. In many cases, cartridge 432 is loaded into chamber 400 of fluid injection system 100. Loading cartridge 432 into fluid injection system 100 can comprise sliding cartridge 432 down chamber 400 with distal end 432a of cartridge 432 oriented closer to the distal end 114 of elongate member 110 than proximal end 432b of cartridge 432. Loading cartridge 432 can comprise contacting cartridge abutment 410 with cartridge plunger 440. In some cases, loading cartridge 432 into fluid injection system 100 comprises engaging cartridge 432 or a portion thereof (e.g., cartridge plunger 440 or plunger interface 442) with cartridge interface 420. Engaging cartridge 432 or a portion thereof with cartridge interface 420 can comprise releaseably engaging cartridge 432 with cartridge interface 420. For example, cartridge 432 or a portion thereof (e.g., cartridge plunger 440 or plunger interface 442) can be punctured by cartridge interface 420 (e.g., wherein cartridge interface 420 comprises a needle or pointed channel) or screwed onto threads of cartridge interface 420. Engaging cartridge 432 or a portion thereof with cartridge interface 420 can comprise establishing a fluidic connection between the fluid 480 contained in cartridge 432 and one or more fluid delivery member 320 (e.g., via delivery channel 270, which may run through cartridge interface 420 and/or cartridge abutment 410). Representative examples of loading cartridge 432 into system 100 are shown in FIG. 4A and FIG. 4B.

At step 1702, at least a portion of fluid injection system 100 (e.g., elongate member 110 or a portion thereof) may be inserted into a tissue (e.g., which may comprise a portion of a subject's body). The dimensions of elongate member 100 (e.g., as disclosed herein) allow for the use of fluid injection system 100 in applications where less invasive interventions would be contraindicated (e.g., wherein a tumor is inoperable and/or wherein systemic intervention might lead to harmful effects, such as an acute immune response). Insertion may occur with one or more fluid delivery members 320 in an unexpanded configuration (e.g., retracted in the elongate member 110 of system 100). In some cases, a disposable or autoclavable coaxial sheath may be positioned around elongate member 110 prior to inserting at least a portion of fluid injection system 100 into a tissue, for example to allow for multiple uses of system 100 (e.g., at different insertion points of a subject's body). In some cases, a coaxial sheath can be anchored to fluid injection system 100 by coupling at least a portion of the coaxial sheath to distal coupling 190.

At step 1703, the distal end of the fluid injection system may be positioned at or near a target tissue (e.g., a tumor or portion thereof within the patient's body). The system may for example be positioned such that elongate member 110 is in close proximity with, for example touching, the target tissue of interest. Positioning the system may for example comprise positioning the system under guidance of an imaging system, for example using an ultrasound or fluoroscopic imaging system.

At step 1704, one or more fluid delivery members 320 may be extended from the distal end 114 of elongate member 110 into the target tissue (e.g., tumor tissue). Extension of fluid delivery members 320 may be actuated by actuator 250, which may comprise a mechanical actuator and/or an electromechanical actuator. Actuator can, for example, comprise a thumbwheel, level, electric actuator, or the like. Actuation of the actuator may be automatic or manual. The plurality of fluid delivery members may be configured to extend out of the distal end of the elongate member with a pre-determined pattern or curvature. The fluid delivery members may be configured to angle away from a longitudinal axis 111 of elongate member 110. The fluid delivery members may for example be configured to angle away from the longitudinal axis 111 of the elongate member at one or more oblique angles relative to the longitudinal axis.

At step 1705, fluid may be injected into the tumor via the fluid delivery members. As disclosed herein, injection of fluid into a target tumor tissue via the fluid delivery members can comprise disengaging actuator 250. In some cases, injecting the fluid into the tumor tissue (e.g., step 1705) can also comprise retracting the fluid delivery members back into the elongate member (e.g., step 1706). For example, some embodiments of fluid injection system 100 can allow for simultaneous injection of the fluid and withdrawal of the fluid delivery members.

At step 1706, the fluid delivery members may be retracted from the tumor into the elongate member. As disclosed herein, fluid delivery members can be retracted (e.g., to an unextended configuration) when actuator 250 is disengaged.

At step 1707, the fluid injection system may be removed from the patient's body.

At step 1708, the tumor may be resected for analysis. The tumor may be resected immediately after fluid injection. The tumor may be resected within 4 hours of fluid injection, for example within 4 to 24 hours, 4 to 48 hours, 6 to 24 hours, or 4 to 8 hours. The tumor may be resected within days of fluid injection, for example within about 1 to about 7 days. The tumor tissue may be analyzed as described herein. For example, the tumor tissue may be analyzed to determine the efficacy of one or more therapeutic agents, or combination of agents, on the tumor.

Although the steps above show a method 1700 of injecting fluid into a tumor within a body of a patient using a fluid injection system in accordance with embodiments, many variations based on the teaching are described herein. The steps may be completed in a different order. Steps may be added or deleted. Some of the steps may comprise sub-steps. Many of the steps may be repeated as often as beneficial or necessary for the desired procedure.

For example, in some embodiments Steps 1705 and 1706 may optionally occur simultaneously such that the fluids are injected into the tumor while the fluid delivery members are slowly retracted back into the elongate member. Simultaneous injection and retraction may for example aid in the formation of injection columns as shown in FIG. 15A to FIG. 15C and as described herein.

In some embodiments, one or more of the steps of the method 1700 may be used for fluid injection into an ex vivo or in vitro tissue. In such embodiments, steps 1702, 1707, and 1708 may be optional in certain embodiments of methods disclosed herein.

Turning to FIGS. 21A-21D, methods disclosed herein can comprise detecting and/or evaluating one or more agents that have been delivered to (e.g., injected into) a tissue prior to any resection or explanting of the injected tissue. Methods disclosed herein can comprise a step comprising inserting at least one fluid delivery member 320 into tissue 702, which can be a tissue of a subject, such as a target tissue comprising a tumor (e.g., as shown in FIG. 21A). Methods disclosed herein can comprise a step comprising delivering (e.g., injecting) one or more agents into the tissue 702 (e.g., as shown in FIG. 21B). Optionally, a method disclosed herein can comprise allowing time to pass, for example, to allow one or more agents delivered to tissue 702 to diffuse or flow through a tissue and/or to allow the one or more agents to affect the tissue 702 (e.g., as shown in FIG. 21C). A radiation source 800 can be used to detect (e.g., via illumination) one or more agents delivered to a tissue 702, (e.g., as shown in FIG. 21D). For example, exact locations 703 of one or more sites at which one or more agents were delivered to a tissue can be quickly and precisely determined by using radiation device 800. In some cases, the ability to detect one or more agents delivered to a tissue 702 can be helpful in determining which tissue(s) or portion(s) of a tissue should be resected (e.g., for analysis), for example, based on a distribution of the one or more agents determined by imaging the tissue 702. While a magnetic detector can be used in addition to or in place of radiation source 702 to detect and/or evaluate one or more agents (e.g., an agent comprising a magnetic tag) delivered to a tissue 702, it will be appreciated by one of skill in the art that a radiation source are capable of more precise determination of spatial distributions of agents and can be used with agents that are not magnetic (e.g., pigmented agents and/or fluorescent agents).

A radiation source 800 can comprise an ultraviolet (UV) light source, a visible light source, an infrared illuminator, or a coherent light source. A radiation source 800 can be a handheld radiation source or a handheld emitter of a larger radiation source, which can allow for detection and/or evaluation of an agent (e.g., an FTM particle) prior to or during a resection or explant procedure. In some cases, a detector, such as a camera or fluorescent light detector can be used to detect a signal of one or more agents delivered to a tissue 702. In many cases, the one or more agent delivered to the tissue (and, optionally, the radiation source 800) will be selected such that the signal from the one or more agent is visible to the naked eye. For example, an FTM particle can be detected using a radiation source 800 prior to resection or explanting of a tissue from a subject (e.g., as disclosed herein).

Steps shown in FIGS. 21A-21D are representative examples of steps that can be included in a method disclosed herein. Some methods disclosed herein do not comprise all steps shown in FIGS. 21A-21D, and some methods disclosed herein may comprise additional steps not shown in FIGS. 21A-21D. For example, a method disclosed herein may comprise detecting one or more agents in a tissue 702 (e.g., one or more fluorescent particles)

### Systems

In some embodiments the system 100 is a handheld system. Alternatively or in combination, the system 100 may be configured for robotic control and operation, for example with instructions from a computer-readable program as described herein.

In some embodiments, the system 100 may be configured as a standalone access device for use in accessing a tissue site of interest, such as a tumor tissue or portion thereof. A fluid injection system 100 comprising an elongate member 110 configured to puncture a subject's skin and/or to penetrate an internal tissue (e.g., a target tissue, such as a cancer tissue) is one of various embodiments of the fluid injection systems 100 disclosed herein that can be configured as a standalone access device. In some cases, a system configured to be a standalone access device can comprise a rounded or pointed tip, which can be useful in piercing or separating biological tissue. In some cases, a system configured to be a standalone access device can comprise a rigid elongate member, which can be useful in manipulating or directing a needle for injection (e.g., one or more fluid delivery members 320) in, through, or around a tissue.

In some embodiments, the system 100 may be configured to be used with conventional non- or minimally-invasive surgical access devices and introducers known to one of ordinary skill in the art. For example, the elongate member 110 may have an outer diameter sized to fit within a working channel of a conventional biopsy access needle, a conventional endoscope, a conventional laparoscopic system, a conventional vascular access sheath, or the like. The system 100 may be inserted into the working lumen of the conventional access device in order to reach the tissue of interest. The versatility of fluid injection systems 100 disclosed herein to be used with existing access devices and needles limits the training that a practitioner will need to become familiar with the use of the fluid injection systems 100 in performing techniques and assays described herein.

Alternatively or in combination, the system 100 may further comprise its own introducer to provide access to the tumor site of interest. For example, a system 100 can comprise a introducer sheath for puncturing or penetrating a tissue. An introducer of system 100 can be coaxial with an axis (e.g., a longitudinal axis) of system 100 or with an axis (e.g., a longitudinal axis) of a component of system 100, such as an elongate member 110. In some cases, an introducer of system 100 is separate from another component of system 100, such as a housing of system 100 and/or an elongate member 110 of system 100. An introducer can be used to create a path to a target tissue (e.g., by inserting the introducer into a tissue of a subject). In some cases, an introducer is used to create a path to a target tissue before another component of system 100, such as an elongate member 110, is inserted into the target tissue and/or any intervening tissue. In some cases, an introducer can be coupled to a distal coupling 190 of system 100 (e.g., wherein distal coupling 190 comprises a Luer lock coupling component).

The devices, systems, and methods described herein may be used in conjunction with an imaging system for peri-operative imaging of the fluid injection system in use. Peri-operative imaging may include imaging of the tumor prior to insertion of the system 100 into the patient, during insertion and positioning of the system 100 adjacent the tumor, during fluid delivery, during retraction and removal of the injection system 100 from the patient, and/or after removal of the system 100. The imaging system may be any imaging system known to one of ordinary skill in the art. For example, the imaging system may be an ultrasound imaging system, an ultrasound biomicroscopy (UBM) system, an X-ray imaging system, a fluorescent imaging system, an Optical Coherence Tomography (OCT) imaging system, a magnetic resonance (MR) imaging system, or any other imaging system known to one of ordinary skill in the art.

Systems or methods disclosed herein may comprise a computer or use thereof. For example, one or more steps of method 1700 (or other method steps disclosed or necessarily implied herein) may be performed by a fully or partially automated system comprising a computer. In some cases, fluid injection system 100 comprises a computer. In some cases, a fluid injection system 100 an imaging system comprises an imaging system (e.g., to aid in insertion, placement and/or actuation of the system). A computer can comprise a processor (e.g., a controller). A computer can comprise a non-transitory computer-readable memory, which can comprise instructions which, when executed, can cause one or more components of the system to perform one or more steps of a method disclosed herein. In some cases, the operation of a system is entirely or partly dependent on one or more user inputs.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A fluid injection system comprising:
an elongate member having a proximal end and a distal end and comprising an inner wall defining a lumen therein;
a plurality of fluid delivery members disposed within the lumen of the elongate member and having a retracted configuration and an extended configuration, wherein the plurality of fluid delivery members are configured to extend out of the distal end of the elongate member in the extended configuration;
wherein each of the plurality of fluid delivery members comprises a distal end, a proximal end, an outlet port at the distal end, and an inner wall defining a fluid delivery lumen therein, the fluid delivery lumen being fluidly coupled to the outlet port,
wherein each of the fluid delivery lumens is fluidly independent of every other fluid delivery lumen of the plurality of fluid delivery members,
a plurality of fluid delivery channels, wherein each of the plurality of fluid delivery channels is fluidly coupled to one or more fluid delivery lumens of the plurality of fluid delivery members; and
a fluid delivery mechanism operably coupled to the plurality of fluid delivery channels, wherein actuation of the fluid delivery mechanism causes fluid to pass from the plurality of fluid delivery channels to the plurality of fluid delivery members and out of the outlet ports.

2. The system of claim 1, wherein actuation of the fluid delivery mechanism is operably coupled to the plurality of fluid delivery members such that delivery of fluid is concomitant with retraction of the fluid delivery members from the extended configuration to the retracted configuration.

3. The system of claim 1 or claim 2, wherein the fluid delivery mechanism comprises a fluid delivery rod.

4. The system of claim 1, wherein the plurality of fluid delivery members are configured to retract from the extended configuration to the retracted configuration simultaneously with fluid delivery from the fluid delivery members.

5. The system of any one of claims 1 to 4, wherein the plurality of fluid delivery members is configured to be fully enclosed within the lumen of the elongate member in the retracted configuration.

6. The system of any one of claims 1 to 5, wherein the elongate member comprises a sheath, a hypotube shaft, or a needle.

7. The system of any one of claims 1 to 6, wherein the elongate member comprises a metal.

8. The system of any one of claims 1 to 7, wherein the elongate member comprises a flexible material.

9. The system of any one of claims 1 to 8, wherein the elongate member comprises a rigid material.

10. The system of any one of claims 1 to 9, wherein the elongate member has a length in a range of about 4 cm to about 250 cm.

11. The system of claim 10, wherein the elongate member has a length in a range of about 4 cm to about 20 cm.

12. The system of claim 10, wherein the elongate member has a length in a range of about 100 cm to about 250 cm.

13. The system of any one of claims 1 to 12, wherein the elongate member has an outer diameter in a range of about 0.9 mm to about 3.5 mm.

14. The system of claim 13, wherein the elongate member has an outer diameter in a range of about 2 mm to about 4 mm.

15. The system of any one of claims 1 to 14, wherein the elongate member has an outer diameter in a range of about 3 French to about 10 French.

16. The system of any one of claims 1 to 15, wherein the elongate member has an outer diameter sized to fit within a working channel of a conventional biopsy access needle, a conventional endoscope, or a conventional vascular access sheath.

17. The system of any one of claims 1 to 16, wherein the elongate member comprises a needle with a gauge number in a range of about 10 to about 20.

18. The system of any one of claims 1 to 17, wherein the plurality of fluid delivery members comprises at least two fluid delivery members.

19. The system of claim 18, wherein the plurality of fluid delivery members comprises from 2 to 20 fluid delivery members.

20. The system of any one of claims 1 to 19, wherein the plurality of fluid delivery members comprises a plurality of needles or tubes.

21. The system of any one of claims 1 to 20, wherein the plurality of fluid delivery members comprises a plurality of pencil-point needles, blunt-tipped needles, or bevel-tipped needles

22. The system of any one of claims 1 to 21, wherein the plurality of fluid delivery members comprises metal or plastic.

23. The system of any one of claims 1 to 22, wherein the plurality of fluid delivery members comprises a shape-memory alloy.

24. The system of any one of claims 1 to 23, wherein the plurality of fluid delivery members comprises a flexible material.

25. The system of any one of claims 1 to 24, wherein the plurality of fluid delivery members comprises a rigid material.

26. The system of any one of claims 1 to 25, wherein each of the plurality of fluid delivery members has an outer diameter in a range of about 0.05 mm to about 0.50 mm.

27. The system of claim 26, wherein each of the plurality of fluid delivery members has an outer diameter of about 0.25 mm.

28. The system of any one of claims 1 to 27, wherein each of the plurality of fluid delivery members is a needle with a gauge number of about 28 to about 33.

29. The system of claim 28, wherein each of the plurality of fluid delivery members is a needle with a gauge number of about 31.

30. The system of any one of claims 1 to 29, wherein each of the fluid delivery lumens of the plurality of fluid delivery members has a volume in a range of about 0.1 μl to about 10 μl.

31. The system of any one of claims 1 to 30, wherein each of the plurality of fluid delivery members has a length extending from the distal end of the elongate member to the proximal end of the elongate member.

32. The system of any one of claims 1 to 31, wherein each of the plurality of fluid delivery members has a length in a range of about 4 cm to about 250 cm.

33. The system of any one of claims 1 to 32, wherein each of the plurality of fluid delivery members has a length extending out of the distal end of the elongate member in the extended configuration in a range of about 5 mm to about 40 mm.

34. The system of any one of claims 1 to 33, wherein each of the plurality of fluid delivery members comprises at least one additional outlet port fluidly coupled to the fluid delivery lumen.

35. The system of any one of claims 1 to 34, wherein, in the extended configuration, each of the plurality of fluid delivery members angle away from a longitudinal axis of the elongate member.

36. The system of claim 35, wherein each of the plurality of fluid delivery members angle away from the longitudinal axis of the elongate member at an angle in a range of about 10° to about 90°.

37. The system of any one of claims 1 to 36, wherein the distal end of the elongate member comprises angling elements positioned to guide the plurality of fluid delivery members to angle away from a longitudinal axis of the elongate member in the extended configuration.

38. The system of any one of claims 1 to 37, wherein, in the extended configuration, each of the plurality of fluid delivery members angle away from a longitudinal axis of the elongate members such that a distance between distal ends of each of the plurality of fluid delivery members is in a range of about 1 mm to about 10 mm.

39. The system of any one of claims 1 to 38, further comprising a handle adjacent to the proximal end of the elongate member.

40. The system of any one of claims 1 to 39, further comprising an actuator adjacent to the proximal end of the elongate member and operably coupled to the plurality of fluid delivery members, wherein actuation of the actuator moves the plurality of fluid delivery members from the retracted configuration to the expanded configuration or from the expanded configuration to the retracted configuration.

41. The system of claim 40, wherein the actuator is configured to retract the plurality of fluid delivery members from the expanded configuration to the retracted configuration at a speed in a range of about 0.1 mm/s to about 10 mm/s.

42. The system of claim 40, wherein the actuator comprises a mechanical actuator or an electromechanical actuator.

43. The system of claim 40, wherein the actuator is manually operated.

44. The system of claim 40, wherein the actuator is automatically operated.

45. The system of claim 40, wherein the fluid delivery mechanism is actuated by the actuator.

46. The system of any one of claims 1 to 45, wherein the fluid delivery mechanism comprises a mechanical actuator or an electromechanical actuator.

47. The system of any one of claims 1 to 46, wherein the fluid delivery mechanism comprises one or more of a plunger or a pump.

48. The system of any one of claims 1 to 47, wherein the fluid delivery mechanism is manually operated.

49. The system of any one of claims 1 to 48, wherein the fluid delivery mechanism is automatically operated.

50. The system of any one of claims 1 to 49, wherein the fluid delivery mechanism is configured to cause fluid to be delivered out of the outlet ports at a flow rate in a range of about 0.1 μl/s to about 10 μl/s.

51. The system of any one of claims 1 to 50, wherein the system is configured for fluid delivery from about 1 cm to about 300 cm below the skin surface.

52. The system of claim 51, wherein the system is configured for fluid delivery from about 1 cm to about 30 cm below the skin surface.

53. The system of claim 51, wherein the system is configured for fluid delivery from about 4 cm to about 20 cm below the skin surface.

54. The system of claim 51, wherein the system is configured for fluid delivery from about 100 cm to about 250 cm below the skin surface.

55. The system of any one of claims 1 to 54, wherein the plurality of fluid delivery channels comprises the fluid delivery lumens of the plurality of fluid delivery members.

56. The system of claim 55, wherein the fluid delivery lumens of the plurality of fluid delivery members are the plurality of fluid delivery channels.

57. The system of any one of claims 1 to 56, wherein the fluid delivery mechanism comprises a plurality of fluid delivery mechanisms, each of the plurality of fluid delivery mechanisms being operably coupled to a single fluid delivery channel of the plurality of fluid delivery channels.

58. The system of any one of claims 1 to 57, further comprising an imaging system for peri-operative imaging of the fluid injection system in use.

59. The system of any one of claims 1 to 58, further comprising one or more cartridges fluidly-coupled to one or more of the fluid delivery lumens or one or more of the plurality of fluid delivery channels.

60. The system of any one of claims 1 to 59, wherein each of the plurality of fluid delivery channels has a volume in a range of about 10 μl to about 500 μl.

61. The system of any one of claims 1 to 60, further comprising a population of fluorescent tracking microspheres (FTM).

62. The system of claim 61, wherein the fluorescent tracking microspheres have a diameter from 5 micrometers to 10 micrometers.

63. The system of claim 61 or claim 62, wherein the fluorescent tracking microspheres comprise polystyrene.

64. The system of any one of claims 1 to 63, further comprising a plurality of populations of fluorescent tracking microspheres (FTM).

65. The system of any one of claims 1 to 64, further comprising a volume selector.

66. The system of any one of claims 1 to 65, further comprising a plurality of cartridges.

67. A method of injecting fluid into a tumor within a body of a patient, the method comprising:
providing a fluid injection system, wherein the fluid injection system comprises an elongate member having a proximal end and a distal end, a plurality of fluid delivery members disposed within a lumen of the elongate member, and a plurality of fluid delivery channels, wherein each of the plurality of fluid delivery channels is fluidly coupled to a single fluid delivery lumen of each of the plurality of fluid delivery members;
inserting the distal end of the elongate member into the body with the plurality of fluid delivery members retracted;
positioning the distal end of the elongate member in close proximity to the tumor with the plurality of fluid delivery members retracted;
extending the plurality of fluid delivery members from the distal end of the elongate member into the tumor; and
injecting a plurality of fluids into the tumor from the plurality of fluid delivery members, wherein each of the plurality of fluid delivery members is fluidly independent from every other of the plurality of fluid delivery members.

68. The method of claim 61, further comprising retracting the plurality of fluid delivery members from the tumor into the distal end of the elongate member.

69. The method of claim 68, wherein retracting the plurality of fluid delivery members occurs concomitantly with injecting the plurality of fluids.

70. The method of claim 68, wherein retracting the plurality of fluid delivery members comprises retracting the plurality of fluid delivery members such that the plurality of fluid delivery members are fully enclosed within the lumen of the elongate member.

71. The method of claim 68, wherein retracting the plurality of fluid delivery members comprises retracting the plurality of fluid delivery members at a speed in a range of about 0.1 mm/s to about 10 mm/s.

72. The method of any one of claims 61 to 71, further comprising removing the distal end of the elongate member from the body with the plurality of fluid delivery members retracted.

73. The method of any one of claims 61 to 72, further comprising resecting at least a portion of the tumor for analysis.

74. The method of any one of claims 61 to 73, further comprising loading the plurality of fluids into the plurality of fluid delivery channels prior to inserting the distal end of the elongate member into the body.

75. The method of any one of claims 61 to 74, further comprising imaging the fluid injection system peri-operatively.

76. The method of any one of claims 61 to 75, wherein the elongate member comprises a sheath, a hypotube shaft, or a needle.

77. The method of any one of claims 61 to 76, wherein the elongate member comprises a metal.

78. The method of any one of claims 61 to 77, wherein the elongate member comprises a flexible material.

79. The method of any one of claims 61 to 78, wherein the elongate member comprises a rigid material.

80. The method of any one of claims 61 to 79, wherein the elongate member has an outer diameter in a range of about 0.9 mm to about 3.5 mm.

81. The method of any one of claims 61 to 80, wherein the elongate member comprises a needle with a gauge number in a range of about 10 to about 20.

82. The method of any one of claims 61 to 81, wherein inserting the distal end of the elongate member into the body comprises inserting the distal end of the elongate member into a working channel of a conventional biopsy access needle, a conventional endoscope, or a conventional vascular access sheath pre-positioned in the body.

83. The method of any one of claims 61 to 82, wherein the plurality of fluid delivery members comprises at least two fluid delivery members.

84. The method of claim 83, wherein the plurality of fluid delivery members comprises from 2 to 20 fluid delivery members.

85. The method of any one of claims 61 to 84, wherein the plurality of fluid delivery members comprises a plurality of needles or tubes.

86. The method of any one of claims 61 to 85, wherein the plurality of fluid delivery members comprises metal or plastic.

87. The method of any one of claims 61 to 86, wherein the plurality of fluid delivery members comprises a shape-memory alloy.

88. The method of any one of claims 61 to 87, wherein the plurality of fluid delivery members comprises a flexible material.

89. The method of any one of claims 61 to 88, wherein the plurality of fluid delivery members comprises a rigid material.

90. The method of any one of claims 61 to 89, wherein each of the plurality of fluid delivery members has an outer diameter of from about 0.05 mm to about 0.50 mm.

91. The method of any one of claims 61 to 90, wherein each of the plurality of fluid delivery members is a needle with a gauge number of about 28 to about 33.

92. The method of any one of claims 61 to 91, wherein injecting the plurality of fluids comprises injecting the plurality of fluids at a flow rate in a range of about 0.1 μl/s to about 10 μl/s.

93. The method of any one of claims 61 to 92, wherein injecting the plurality of fluids comprises injecting, from each of the plurality of fluid delivery members, a volume in a range of about 10 μl to about 500 μl of each of the plurality of fluids.

94. The method of any one of claims 61 to 93, wherein each of the plurality of fluid delivery members has a length extending from the distal end of the elongate member to the proximal end of the elongate member.

95. The method of any one of claims 61 to 94, wherein each of the plurality of fluid delivery members has a length in a range of about 4 cm to about 250 cm.

96. The method of any one of claims 61 to 95, wherein extending the plurality of fluid delivery members comprises extending a length in a range of about 5 mm to about 40 mm of each of the plurality of fluid delivery members out of the distal end of the elongate member into the tumor.

97. The method of any one of claims 61 to 96, wherein extending the plurality of fluid delivery members comprises extending the plurality of fluid delivery members from the distal end of the elongate member such that the plurality of fluid delivery members angle away from a longitudinal axis of the elongate member.

98. The method of any one of claims 61 to 97, wherein the distal end of the elongate member comprises angling elements positioned to guide the plurality of fluid delivery members to angle away from the longitudinal axis of the elongate member in the extended configuration.

99. The method of any one of claims 61 to 98, wherein injecting the plurality of fluids comprises creating a plurality of distinct fluid columns in the tumor.

100. The method of any one of claims 61 to 99, wherein the fluid injection system further comprises a handle having a fluid delivery mechanism thereon, the fluid delivery mechanism being operably coupled to the plurality of fluid delivery channels, and wherein injecting the plurality of fluids comprises actuating the fluid delivery mechanism.

101. The method of claim 100, wherein actuating the fluid delivery mechanism comprises manually actuating the fluid delivery mechanism.

102. The method of claim 100, wherein actuating the fluid delivery mechanism comprises automatically actuating the fluid delivery mechanism.

103. The method of claim 100, wherein the fluid delivery mechanism comprises a mechanical actuator or an electromechanical actuator.

104. The method of claim 100, wherein the fluid delivery mechanism comprises one or more of a plunger or a pump.

105. The method of any one of claims 61 to 104, wherein the fluid injection system further comprises an actuator adjacent to the proximal end of the elongate member and operably coupled to the plurality of fluid delivery members, and wherein extending the plurality of fluid delivery members comprises actuating the actuator.

106. The method of claim 105, wherein actuating the actuator comprises manually actuating the actuator.

107. The method of claim 105, wherein injecting the plurality of fluids comprises actuating the actuator.

108. The method of claim 105, wherein actuating the actuator comprises automatically actuating the actuator.

109. The method of claim 105, wherein the actuator comprises a mechanical actuator or an electromechanical actuator.

110. The method of claim 105, wherein the actuator comprises one or more of a thumbwheel or an electric actuator.

111. The method of any one of claims 61 to 110, wherein injecting the plurality of fluids comprises injecting the plurality of fluids from about 0.2 cm to about 20 cm below the skin surface.

112. The method of claim 111, wherein injecting the plurality of fluids comprises injecting the plurality of fluids from about 1 cm to about 30 cm below the skin surface.

113. The method of claim 111, wherein injecting the plurality of fluids comprises injecting the plurality of fluids from about 4 cm to about 20 cm below the skin surface.

114. The method of claim 111, wherein injecting the plurality of fluids comprises injecting the plurality of fluids from about 100 cm to about 250 cm below the skin surface.

115. The method of any one of claims 61 to 114, wherein the plurality of fluids comprises one or more therapeutic agents.

116. The method of any one of claims 61 to 115, wherein injecting the plurality of fluids comprises injecting a different fluid from each of the plurality of fluid delivery members into the tumor.

117. The method of any one of claims 61 to 116, wherein injecting the plurality of fluids comprises injecting a same fluid from each of the plurality of fluid delivery members into the tumor.

118. The method of any one of claims 61 to 117, wherein the tumor is located in the skin, breast, brain, prostate, colon, rectum, kidney, pancreas, lung, liver, heart, stomach, intestines, ovaries, testes, cervix, lymph nodes, thyroid, esophagus, head or neck, eye, bone, or bladder of the patient.

119. The method of any one of claims 61 to 117, wherein the plurality of fluids comprises a population of fluorescent tracking microspheres (FTM).

120. The method of any one of claims 67 to 119, wherein the plurality of fluids comprises a plurality of populations of fluorescent tracking microspheres.
